(19) United States
(12) Patent Application Publication
(10) Pub. No.: US 2021/0090191 A1
(43) Pub. Date: Mar. 25, 2021

(54) SYSTEM AND METHODS FOR ESTATE PLANNING PREDICTIONS AND RECOMMENDATIONS

(71) Applicant: Wells Fargo Bank, N.A., San Francisco, CA (US)

(72) Inventors: Tracy W. Chan, San Francisco, CA (US); Haritha Devarajan, Dublin, CA (US); Ellen Fabini, San Francisco, CA (US); Jennifer Greene, Novato, CA (US); Darius Miranda, San Francisco, CA (US); Marria Rhodriquez, San Francisco, CA (US)

(21) Appl. No.: 15/603,083

(22) Filed: May 23, 2017

Related U.S. Application Data
(60) Provisional application No. 62/491,293, filed on Apr. 28, 2017.

(51) Int. Cl. G06Q 50/18; G06N 5/02

(52) U.S. Cl. CPC G06Q 50/186; G06N 5/022

(57) ABSTRACT

A system, method, and apparatus for offering estate planning prediction and recommendation services to a client using a server system including a processor and a memory having instructions stored in non-transitory machine-readable media, the instructions executable by the processor, is provided. The server system is structured to receive information relating to one or more assets of a client, monitor a health status of the client, determine a redistribution plan for the one or more assets of the client, based on the health status of the client, and redistribute the one or more assets of the client according to the redistribution plan.

### CROSS-REFERENCE TO RELATED APPLICATIONS

This application claims the benefit of priority to U.S. application Ser. No. 62/491,293, filed Apr. 28, 2017, entitled “SYSTEM AND METHODS FOR ESTATE PLANNING PREDICTIONS AND RECOMMENDATIONS”, which is hereby incorporated by reference in its entirety.

### TECHNICAL FIELD

Embodiments of the present disclosure relate generally to the field of estate planning.

### BACKGROUND

Because estate planning laws can be complicated and opaque, many individuals do not understand how estate planning works or why estate planning is important. These individuals often also do not otherwise possess the resources to understand estate planning and what options are most beneficial to them or most in line with their interests. Accordingly, an estate planning system that automatically determines beneficiaries and asset distribution plans based on an individual's biographical information and interests would be beneficial to many individuals.

Additionally, many individuals procrastinate making estate plans because estate planning can be time-consuming or unpleasant. However, procrastinating estate planning decisions often leads to end-of-life financial complications for individuals, their relatives, and their intended beneficiaries. As such, an estate planning system that monitors an individual's health and, based on that health information, automatically determines asset redistribution plans and estate planning issues the individual should address in order to settle end-of-life financial issues would further be beneficial to many individuals.

### SUMMARY OF THE INVENTION

One embodiment relates to a system. The system includes a server system including a processor and a memory having instructions stored in non-transitory machine-readable media, the instructions executable by the processor. The server system is structured to receive information relating to one or more assets of a client and monitor a health status of the client. The server system is further structured to determine a redistribution plan for the one or more assets of the client, based on the health status of the client, and redistribute the one or more assets of the client according to the redistribution plan.

Another embodiment relates to a method. The method includes receiving, by a financial institution computing system, information relating to one or more assets of a client and monitoring, by the computing system, a health status of the client. The method also includes determining, by the computing system, a redistribution plan for the one or more assets of the client, based on the health status of the client. The method further includes redistributing, by the computing system, the one or more assets of the client according to the redistribution plan.

Another embodiment relates to an apparatus. The apparatus includes a network interface structured to communicate data to and from a server system associated with a financial institution, a display configured to present information to a client, an input/output device structured to exchange data with the client, and a processing circuit including a processor and a memory. The memory is structured to store instructions that are executable by the processor and cause the processing circuit to receive, by the input/output device, information relating to one or more assets of the client and transmit, by the network interface, the information on the one or more assets of the client to the server system. The memory also causes the processing circuit to gather, by at least the input/output device, information relating to health of the client and transmit, by the network interface, the information relating to the health of the client to the server system. The memory further causes the processing circuit to receive, by the network interface, a redistribution plan for the one or more assets of the client from the server system, the redistribution plan based on a health status of the client determined by the server system from the transmitted health information; capture, by the input/output device, one or more client selections relating to the redistribution plan; and transmit, by the network interface, the one or more client selections to the server system.

### BRIEF DESCRIPTION OF THE DRAWINGS

FIG. 1 is an environmental view of an estate planning prediction and recommendation system, according to an example embodiment.

FIG. 2 is a detailed schematic diagram of a financial institution computing system of FIG. 1, according to an example embodiment.

FIG. 3 is a flow diagram illustrating a method of providing estate planning predictions and recommendations, by the financial institution computing system of FIGS. 1 and 2, according to an example embodiment.

FIG. 4 is a flow diagram illustrating a method of providing beneficiary recommendations based on life events, by the financial institution computing system of FIGS. 1 and 2, according to an example embodiment.

FIGS. 5-9 is are images of graphical user interfaces used in connection with the flow diagrams of FIGS. 3 and 4, according to example embodiments.

### DETAILED DESCRIPTION

Referring to the Figures generally, various systems, methods, and apparatuses for providing estate planning predictions and recommendations are described herein. More particularly, systems and methods for monitoring a client's health and recommending a distribution of the client's assets based on the client's monitored health are described herein. Additionally, the systems and methods described herein are directed to recommending beneficiaries for the client based on the client's biographical information and/or based on the client's interests. The systems and methods described herein are further directed to tracking “life events” of the client (e.g., births, marriages, deaths, etc.) and modifying the client's beneficiaries based on the tracked life events.

An example implementation is described as follows. A client sends a request to a financial institution to access estate planning prediction and recommendation services provided by the financial institution. Once the request of the client has been approved, a computing system associated with the financial institution gathers information about the client, the client's interests, the client's assets, the client's asset distribution preferences, and so on. In some arrangements, the client additionally grants the computing system access to the client's assets such that the financial institution computing system is able to automatically redistribute the client's assets and/or automatically modify beneficiaries for the client. The computing system also monitors the physical and/or mental health of the client. If the computing system senses a decline in the client's health (e.g., the computing system determines that the client is in poor health), the computing system notifies the client to carry out estate planning activities and/or recommends a redistribution of the client's assets. In some embodiments, the computing system further automatically redistributes the client's assets in response to declining health of the client.

If the client's assets are redistributed, either automatically by the computing system or manually by the client, such that the redistributed assets include a higher percentage earmarked for donation, the computing system recommends to the client various charities, relatives, organizations, etc. that the client can set as beneficiaries of the increased donation percentage. The computing system recommends the potential beneficiaries to the client based on the client's biographical information and/or the client's interests. In some arrangements, in recommending potential beneficiaries to the client, the financial institution computing system includes individuals or organizations hoping to be funded by one or more clients of the financial institution's estate planning prediction and recommendation services (“submitters”). To be considered as beneficiaries, each submitter provides a submission to the computing system indicating the same. Each submission describes the reason(s) the submitting individual or organization hopes to obtain funding, what the individual or organization hopes to do with the funding, etc. In some arrangements, the computing system provides to the client the recommended potential beneficiaries (e.g., provides the submissions associated with the recommended submitters to the client) so that the client may select beneficiaries from the recommended potential beneficiaries. Alternatively, in other arrangements, the computing system automatically sets one or more of the recommended potential beneficiaries as actual beneficiaries of the client's assets.

In addition to monitoring the physical health of the client, the financial institution computing system also monitors for various events in the life of the client (e.g., births, marriages, deaths). In response to detecting a life event for the client (e.g., determining that the client has gotten married), the financial institution computing system recommends modifying, or automatically modifies, the beneficiaries for the client's assets. In addition, in response to detecting a life event for the client, in some arrangements the computing system also recommends modifying the client's asset distributions similarly as described above.

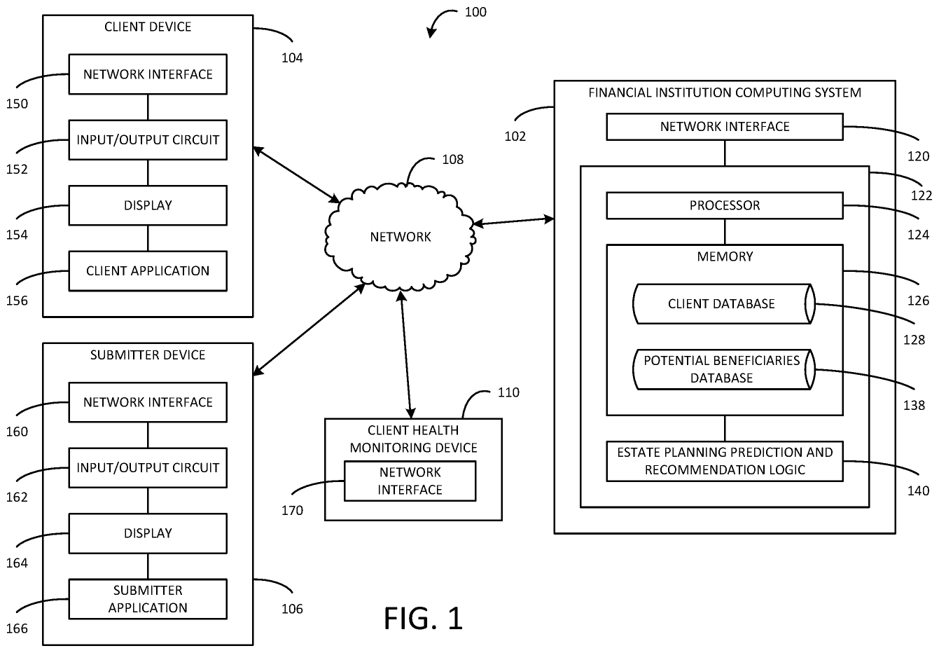

Referring now to FIG. 1, an environmental view of an estate planning prediction and recommendation system 100 is shown, according to an example embodiment. As described in further detail below, the system 100 includes a financial institution computing system 102, a client device 104, and a submitter device 106 connected by a secure network (e.g., network 108). In some embodiments, the system 100 further includes a client health monitoring device 110 connected to the network 108.

The financial institution computing system 102 is associated with or operated by a financial institution (e.g., a bank or a credit card issuer) or any other entity interested in offering estate planning prediction and recommendation services. In practice, the financial institution computing system 102 includes server computer systems, for example, comprising one or more networked computer servers together having at least a processor and non-transitory machine readable media with instructions stored thereon.

As shown in FIG. 1, the financial institution computing system 102 includes a network interface 120, which is used to establish connections with other components of the system 100 by way of network 108. The network interface 120 includes program logic that facilitates connection of the financial institution computing system 102 to the network 108. The network interface 120 supports communication between the financial institution computing system 102 and other systems, such as the client device 104, the submitter device 106, and/or the client health monitoring device 110. For example, the network interface 120 includes any of a cellular transceiver (Code Division Multiple Access (CDMA), Global System for Mobile Communications (GSM), Long-Term Evolution (LTE), etc.), a wireless network transceiver (e.g., 802.11X, ZigBee, or Bluetooth), or a combination thereof (e.g., both a cellular transceiver and a Bluetooth transceiver). In some arrangements, the network interface 120 includes hardware and machine-readable media sufficient to support communication over multiple channels of data communication. Further, in some arrangements, the network interface 120 includes cryptography capabilities to establish a secure or relatively secure communication session with the client device 104, the submitter device 106, the client health monitoring device 110, and/or another device in communication with the financial institution computing system 102. In this regard, personal information about clients, financial data, and other types of data is encrypted and transmitted to prevent or substantially prevent the threat of hacking.

The financial institution computing system 102 also includes a processing circuit 122 having a processor 124 and a memory 126. The processor 124 is implemented as a general-purpose processor, an application specific integrated circuit (ASIC), one or more field programmable gate arrays (FPGAs), a digital signal processor (DSP), a group of processing components, or other suitable electronic processing components. The one or more memory devices 126 (e.g., RAM, NVRAM, ROM, Flash Memory, and/or hard disk storage) store data and/or computer code for facilitating the various processes described herein. Moreover, in some arrangements, the one or more memory devices 126 are or include tangible, non-transient volatile memory or non-volatile memory. Accordingly, in some arrangements, the one or more memory devices 126 include database components, object code components, script components, or any other type of information structured for supporting the various activities and information structures described herein.

The financial institution computing system 102 further includes a client database 128 and a potential beneficiaries database 138. The client database 128 is configured to retrievably store information relating to clients of the financial institution computing system 102. To begin with, the client database 128 stores client biographical information and interests. The client database 128 also stores assets information, as well as the permissions given by each client to the financial institution computing system 102 to access and/or manage the client's assets. The client database 128 further stores beneficiary information for clients and the distribution preferences that clients have for their respective assets. The potential beneficiaries database 138 is configured to retrievably store information about individuals, groups, organizations, etc. that the financial institution computing system 102 recommends to various clients as “potential beneficiaries.” Potential beneficiaries are recommended to clients based on commonalities between the clients' biographical information and/or interests and characteristics (e.g., traits, goals, causes, locations, or affiliations) of the individuals, groups, organizations, etc., which are also stored in the potential beneficiaries database 138. Additionally, in some arrangements, the information stored in the potential beneficiaries database 138 includes submissions made by submitters (e.g., individuals, groups, and/or organizations) hoping to receive funding from one or more clients of the financial institution computing system for various projects, aspirational goals, educational goals, charitable goals, etc.

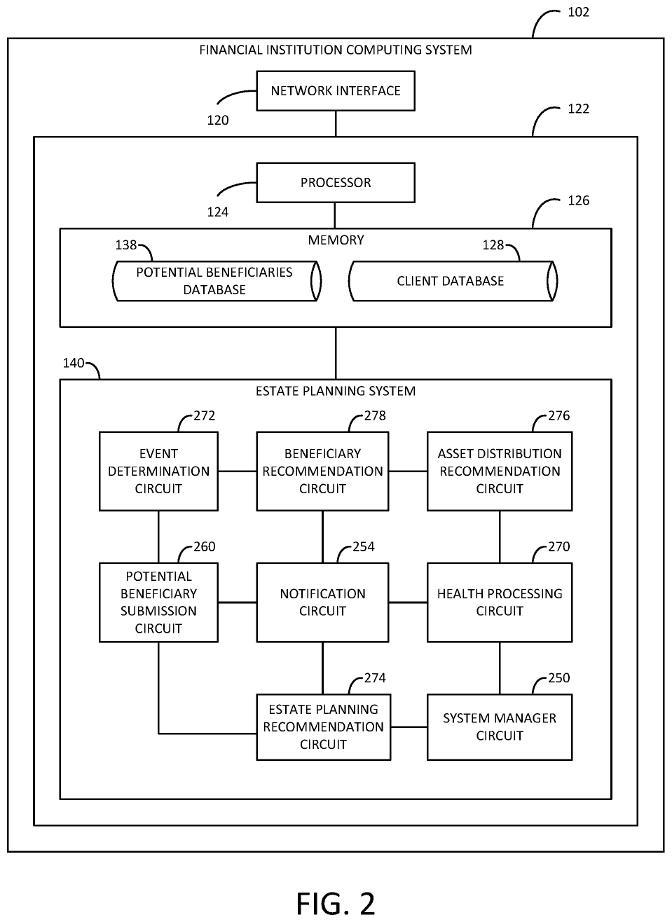

The financial institution computing system 102 further includes an estate planning system 140. The financial institution computing system 102 uses the estate planning system 140 to perform various functions related to estate planning, such as monitoring the client's physical and/or mental health, recommending that the client complete certain estate planning activities, recommending asset distributions, and recommending potential beneficiaries. The estate planning system 140 is described in further detail below with respect to FIG. 2.

As shown in FIG. 1, the system 100 also includes a client device 104. The client device 104 is used by a client to provide information to the financial institution computing system 102. Additionally, the client uses the client device 104 to communicate with the financial institution computing system 102 regarding the distribution of the client's assets, the beneficiaries for one or more of the client's assets, and so on. The client device 104 includes any type of mobile device or any other computing device that is used by a client in connection with services provided by a financial institution. As such, the client device 104 includes, but is not limited to, a phone (e.g., a smartphone), a computing device (e.g., a tablet computer, a laptop computer, a desktop computer, or a personal digital assistant), a wearable device (e.g., a smart watch, smart glasses, or a smart bracelet), and so on.

In various embodiments, the client device 104 includes a network interface 150, an input/output circuit 152, a display 154, and a client application 156. The network interface 150 of the client device 104 is adapted for and configured to establish a communication session via the network 108 with the financial institution computing system 102. Accordingly, the network interface 150 includes any of a cellular transceiver (Code Division Multiple Access (CDMA), Global System for Mobile Communications (GSM), Long-Term Evolution (LTE), etc.), a wireless network transceiver (e.g., 802.11X, ZigBee, or Bluetooth), or a combination thereof (e.g., both a cellular transceiver and a Bluetooth transceiver).

The input/output circuit 152 is structured to receive and provide communication(s) to a client using the client device 104. In this regard, the input/output circuit 152 is structured to exchange data, communications, instructions, etc. with input/output components of the client device 104. Accordingly, in one embodiment, the input/output circuit 152 includes an input/output device such as a touchscreen, a keyboard, a microphone, or a speaker. In another embodiment, the input/output circuit 152 includes communication circuitry for facilitating the exchange of data, values, messages, and the like between an input/output device and the components of the client device 104. In yet another embodiment, the input/output circuit 152 includes machine-readable media for facilitating the exchange of information between the input/output device and the components of the client device 104. In still another embodiment, the input/output circuit 152 includes any combination of hardware components (e.g., a touchscreen), communication, circuitry, and machine-readable media.

In various embodiments, the display 154 is a screen, a touch screen, a monitor, etc. The client device 104 uses the display 154 to communicate information to the client (e.g., by displaying the information to the client on the display 154) and/or to receive communications from the client (e.g., through a keyboard provided on a touchscreen displayed on the display 154). In some embodiments, the display 154 is a component in an input/output device, as described above.

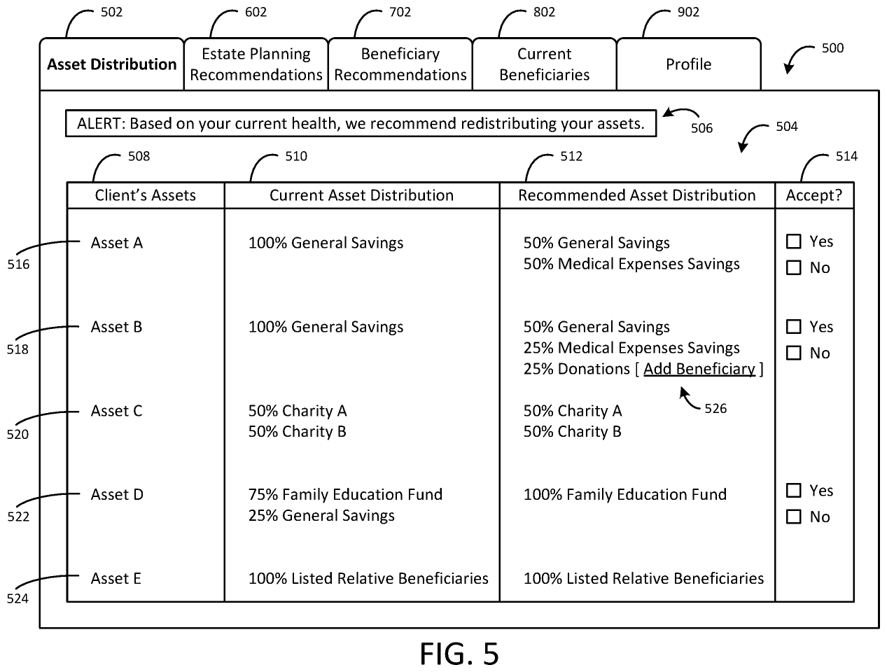

The client application 156 is configured to allow the client to provide information related to estate planning services, such as information about the client, about the client's assets, and about the client's beneficiaries, to the financial institution computing system 102. The client application 156 is further configured to allow the client to receive communications from the computing system 102, such communications relating to recommended estate planning activities, recommended asset distribution plans, and recommended beneficiaries. As such, the client application 156 is communicably coupled to the financial institution computing system 102 via the network 108. The client application 156 includes program logic (i.e., stored executable instructions) configured to implement at least some of the functions described herein. Accordingly, in one example, the client application 156 is a mobile banking application for a smartphone. In another example, the client application 156 is simply a web browser (e.g., Internet Explorer®, Chrome®, or Safari®) configured to receive and display web pages received from the financial institution computing system 102. In other arrangements, the client application 156 includes a text message interface, or another program suitable for communicating with the financial institution computing system 102 over the network 108. As described in further detail below, for example, the client device 104 receives and displays screens (e.g., on the display 154) via the client application 146, the screens including information about the client's assets, recommendations for distribution of the client's assets, recommendations for beneficiaries for the client, and so on (e.g., as shown in FIGS. 5-9, as discussed below).

As shown in FIG. 1, the system 100 further includes a submitter device 106. The submitter device 106 is used by an individual or organization providing a submission (i.e., by a “submitter”) to the financial institution computing system 102, the submission including reasons the individual or organization would like to receive funding as a beneficiary of one or more clients of the financial institution computing system 102. For example, the submitter describes in the submission how the submitter would, if selected as a beneficiary, use provided funds to do charitable work, perform research, accomplish educational goals, etc. The submission is stored in the potential beneficiaries database 138, as described above, and matched with one or more clients based on commonalities between the clients' biographical information and/or interests, and the submitter and/or the associated submission. Once a submitter is recommended to a client, the client may choose to select the submitter as a beneficiary, or the computing system 102 automatically sets the submitter as a beneficiary. In this way, the submitter is able to receive funding via the financial institution computing system 102 from one or more clients.

Similar to the client device 104, the submitter device 106 includes any type of mobile device or any other computing device operated by a submitter in connection with services provided by a financial institution. As such, the submitter device 106 includes, but is not limited to, a phone (e.g., a smartphone), a computing device (e.g., a tablet computer, a laptop computer, a desktop computer, or a personal digital assistant), a wearable device (e.g., a smart watch, smart glasses, or a smart bracelet), and so on.

In various embodiments, the submitter device 106 includes a network interface 160, an input/output circuit 162, a display 164, and a submitter application 166, similar to the client device 104. The network interface 160 of the submitter device is adapted for and configured to establish a communication session via the network 108 with the financial institution computing system 102. Accordingly, the network interface 160 includes any of a cellular transceiver (Code Division Multiple Access (CDMA), Global System for Mobile Communications (GSM), Long-Term Evolution (LTE), etc.), a wireless network transceiver (e.g., 802.11X, ZigBee, or Bluetooth), or a combination thereof (e.g., both a cellular transceiver and a Bluetooth transceiver).

The input/output circuit 162 is structured to receive and provide communication(s) to a submitter using the submitter device 106. In this regard, the input/output logic 162 is structured to exchange data, communications, instructions, etc. with an input/output component of the submitter device 106. Accordingly, in one embodiment, the input/output circuit 162 includes an input/output device such as a touchscreen, a keyboard, a microphone, or a speaker. In another embodiment, the input/output circuit 162 includes communication circuitry for facilitating the exchange of data, values, messages, and the like between an input/output device and the components of the submitter device 106. In yet another embodiment, the input/output circuit 162 includes machine-readable media for facilitating the exchange of information between the input/output device and the components of the submitter device 106. In still another embodiment, the input/output circuit 162 includes any combination of hardware components (e.g., a touchscreen), communication circuitry, and machine-readable media.

In various embodiments, the display 164 is a screen, a touch screen, a monitor, etc. The submitter device 106 uses the display 164 to communicate information to the submitter (e.g., by displaying the information to the submitter on the display 164) and/or to receive communications from the submitter (e.g., through a keyboard provided on a touchscreen displayed on the display 164). In some embodiments, the display 164 is a component in an input/output device, as described above.

The submitter application 166 is configured to allow the submitter to create and provide a submission to the financial institution computing system 102. As such, the submitter application 166 is communicably coupled to the financial institution computing system 102 via the network 108. The submitter application 166 includes program logic (i.e., stored executable instructions) configured to implement at least some of the functions described herein. Accordingly, in one example, the submitter application 166 is a mobile banking application for a smartphone. In another example, the submitter application 166 is simply a web browser (e.g., Internet Explorer®, Chrome®, or Safari®) configured to receive and display web pages received from the financial institution computing system 102. In other arrangements, the submitter application 166 includes a text message interface, or another program suitable for communicating with the financial institution computing system 102 over the network 108. As described in further detail below, for example, the submitter device 106 receives and displays (e.g., on the display 164) screens via the submitter application 166, the screens including information about providing submissions to the financial institution computing system 102, and so on. In some embodiments, the submitter application 166 is the same application as or a similar application to the client application 156. As such, for example, the submitter application 166 and the client application 156 are both a mobile banking application, with different functionalities of the mobile banking application providing services to clients and submitters. In other embodiments, the submitter application 166 is different from the client application 156.

In some embodiments, the estate planning prediction and recommendation system 100 further includes a client health monitoring device 110. The client health monitoring device 110 is any device or system configured to monitor the physical and/or mental health of the client. In some arrangements, the client health monitoring device 110 is an activity tracker (e.g., a Fitbit® or a fitness watch), a pedometer, a body scale, a smart watch, a smartphone, an implantable medical device, an external medical device, and so on. In other arrangements, the client health monitoring device 100 is any device by which the financial institution computing system 102 receives information about the client's physical and/or mental health. In one example, assuming the client provides permission, the client health monitoring device 110 is a computer system (e.g., a desktop computer, a smart phone, a smart tablet, a laptop computer, or a smart watch) by which a health professional associated with the client (e.g., a primary care physician, a nurse, a medical specialist, or a surgeon) communicates health information about the client to the financial institution computing system 102. The health information includes, for example, e-medical records for the client, a health rating for the client determined by the health professional, and/or information about a hospital stay of the client. In another example, the client health monitoring device 110 is a computer system (e.g., a desktop computer, a smart phone, a smart tablet, a laptop computer, or a smart watch) by which a healthcare facility associated with the client (e.g., a health clinic, a hospital, or a patient care facility) communicates health information about the client to the financial institution computing system 102.

As shown in FIG. 1, the client health monitoring device 110 includes a network interface 170. The network interface 170 is adapted for and configured to establish a communication session via the network 108 with the financial institution computing system 102. Accordingly, the network interface 170 includes any of a cellular transceiver (Code Division Multiple Access (CDMA), Global System for Mobile Communications (GSM), Long-Term Evolution (LTE), etc.), a wireless network transceiver (e.g., 802.11X, ZigBee, or Bluetooth), or a combination thereof (e.g., both a cellular transceiver and a Bluetooth transceiver). In some embodiments, the network interface 170 is a local device that the client health monitoring device 110 connects to and which then establishes a connection between the client health monitoring device 110 and the network 108. Accordingly, in one example, the client health monitoring device 110 is an implantable medical device that lacks the capability to directly connect with the network 108. Instead, the implantable medical device connects with a local device, (i.e., the network interface 170) using Bluetooth pairing, RFID waves, etc. The local device/network interface 170 then serves as an intermediary, connecting the implantable medical device with the network 108. In various embodiments, the client health monitoring device 110 includes other components or functionalities, such as an input/output circuit, an input/output device, a display, one or more processors, one or more memories, and so on. In some embodiments, the client health monitoring device 110 also includes one or more components for monitoring the health of the client using the device 110, such as a heart rate monitor, a blood pressure monitor, an accelerometer, a scale, a body mass index (BMI) calculator, and so on.

Referring now to FIG. 2, a detailed schematic diagram of the financial institution computing system 102 is shown, according to an example embodiment. As described above with respect to FIG. 1, the financial institution computing system 102 includes the network interface 120 and the processing circuit 122. The processing circuit 122 further includes the processor 124, the memory 126, and the estate planning system 140, and the memory 126 includes the client database 128 and the potential beneficiaries database 138. As shown in FIG. 2, the estate planning system 140 includes a system manager circuit 250, a notification circuit 254, a potential beneficiary submission circuit 260, a health processing circuit 270, an event determination circuit 272, an estate planning recommendation circuit 274, an asset distribution recommendation circuit 276, and a beneficiary recommendation circuit 278.

The system manager circuit 250 is structured to facilitate communication between the financial institution computing system 102 and the client device 104. To begin with, the system manager circuit 250 receives and processes requests from clients for estate planning prediction and recommendation services. In some arrangements, the client is a new client of the financial institution associated with the financial institution computing system 102. The new client signs up through the financial institution (e.g., via the system manager circuit 250) to receive the estate planning prediction and recommendation services offered by the computing system 102. In other arrangements, the client is an existing client of the financial institution who signs up through the financial institution (e.g., via the system manager circuit 250) to also receive the estate planning prediction and recommendation services offered by the financial institution computing system 102.

In some embodiments, the estate planning prediction and recommendation services are offered to the client for free by the financial institution computing system 102. In one example, the financial institution provides the estate planning prediction and recommendation services, through the computing system 102, to all clients of the financial institution for free, to clients with a certain amount of money in accounts with the financial institution for free, and so on. However, in other embodiments, the estate planning prediction and recommendation services are offered to the client on a fee basis (e.g., the client is charged a fee when the client uses the estate planning prediction and recommendation services), on a subscription basis (e.g., the client pays the financial institution a certain amount of money per week, per month, or per year to access the estate planning prediction and recommendation services), or so on. Accordingly, in fee-based embodiments, the system manager circuit 250 is further structured to collect the fee from the client.

The system manager circuit 250 is also structured to gather and retrievably store certain information from the client that the financial institution computing system 102 uses in providing estate planning services to the client. To begin with, the system manager circuit 250 gathers biographical information about the client and information about the client's interests. In one embodiment, the system manager circuit 250 gathers the biographical and interests information directly from the client. As an example, the system manager 250 asks for and receives client inputs (e.g., via the client device 104) indicating the client's biographical information and interests. In another embodiment, if the client is an existing client of the financial institution, the system manager circuit 250 gathers at least some of the biographical and interests information from a profile the financial institution already has on the client (e.g., from client information associated with other accounts the client holds with the financial institution). In a third embodiment, the system manager circuit 250 gathers the biographical and interests information from third parties. As an example, the system manager circuit 250 gathers information about the client and/or about the client's interests from social media (e.g., by using Application Programming Interfaces (API) to interface with one or more social media platforms). As another example, the system manager circuit 250 gathers information about the client by performing an internet search on the client.

The biographical information gathered on the client includes, for example, the client's name, address, birthday, associated educational institutions (e.g., a high school, college, graduate school, or professional school that the client attended), degree(s), current or former profession(s), membership in various organizations, hometown, religious affiliation, political affiliation, and so on. The interests information gathered for the client includes, for example, any activities, hobbies, causes, research, and so on that the client expresses interest in. In one embodiment, the system manager circuit 250 gathers the interests information by providing a predetermined interests list to the client (e.g., via the client device 104), and the client provides to the system manager circuit 250 the client's interests by selecting from the predetermined interests list. In another embodiment, the client provides to system manager circuit 250 the client's interests in a more freeform manner (e.g., the client types various interests into an input box, and the system manager circuit 250 matches those interests to recognized interests in a database of interests).

In some embodiments, the system manager circuit 250 gathers from the client a ranking of the client's biographical and/or interests information or another indication from the client as to which of the client's biographical and/or interests information is most important to the client. As an example, assume a client provides to the system manager circuit 250 biographical information including the client's address, the undergraduate university the client attended, the graduate university the client attended, the degrees the client has earned, and the client's political affiliation. The client also informs the system manager circuit 250 that the client's interests include golf, helping the elderly, helping the environment, and animal rights. The system manager circuit 250 then asks the client which of this entered information is most important to the client, and the client instructs the system manager circuit 250 that the most important information is the client's undergraduate university, helping the elderly, and animal rights. Subsequently, the computing system 102 prioritizes this information in recommending potential beneficiaries for the client, as described in further detail below.

Moreover, in various embodiments, the system manager circuit 250 gathers and stores additional information about or from the client regarding the client's assets. In certain embodiments, the additional information includes information about the client's assets, information about current or intended beneficiaries for one or more of the client's assets, and/or the client's asset distribution preferences. Client assets include the client's checking accounts, savings accounts, trusts, stocks, bonds, derivatives, property, real estate income, insurance policies, and so on. In one embodiment, the system manager circuit 250 asks the client directly for information about the client's assets and beneficiaries. In another embodiment, the system manager circuit 250, additionally or alternatively, searches public records to find client assets (e.g., to find property that the client owns) and/or beneficiaries. Additionally, in certain embodiments, the system manager circuit 250 asks the client for permission to access the client's assets such that the financial institution computing system 102 is able to monitor the assets. For example, the client provides to the financial institution computing system 102, through the system manager circuit 250, permission to monitor the client's savings accounts so that the computing system 102 can determine when deposits and withdrawals have been made to the savings accounts. Furthermore, in other embodiments, the system manager circuit 250 asks the client for permission to access and manage the client's assets such that the financial institution computing system 102 is able to monitor the assets and redistribute them and/or to modify the beneficiaries of the assets, as described in further detail below.

The client's asset distribution preferences include client preferences the financial institution computing system 102 should take into account when determining a redistribution plan for the client's assets. Accordingly, examples of asset distribution preferences include in response to declining health, saving more money for medical expenses, increasing a donation percentage, saving more money for family members' education, saving more money for family members' living expenses, and adding more assets to a trust. In some arrangements, asset distribution preferences also include whether, for example, the client wants the financial institution computing system 102 (a) to automatically redistribute some or all of the client's assets after determining an asset redistribution plan or simply provide the plan to the client, and/or (b) to automatically set one or more recommended potential beneficiaries as actual beneficiaries of certain client asserts or simply provide the list of recommended potential beneficiaries to the client. In one embodiment, the system manager circuit 250 asks the client for asset distribution preferences once the system manager circuit 250 has received asset information for the client. In another embodiment, the system manager circuit 250 asks the client for asset distribution preferences once the asset distribution recommendation circuit 276 has determined that the client's assets should be redistributed, as described in further detail below.

As shown in FIG. 2, the system manager circuit 250 is communicably and operatively coupled to the client database 128. Accordingly, the system manager circuit 250 stores the biographical information about the client and the interests information for the client, along with any rankings or prioritizations of the client's biographical and/or interests information received from the client, in the client database 128. The system manager circuit 250 also stores gathered information about the client's assets, information about the client's beneficiaries, and the client's asset distribution preferences in the client database 128. Those of skill in the art will appreciate, however, that in various embodiments the system manager circuit 250 is structured to gather other information about the client and/or the client's assets not described herein and store this additional information in the memory 126 (e.g., in the client database 128 or in additional databases).

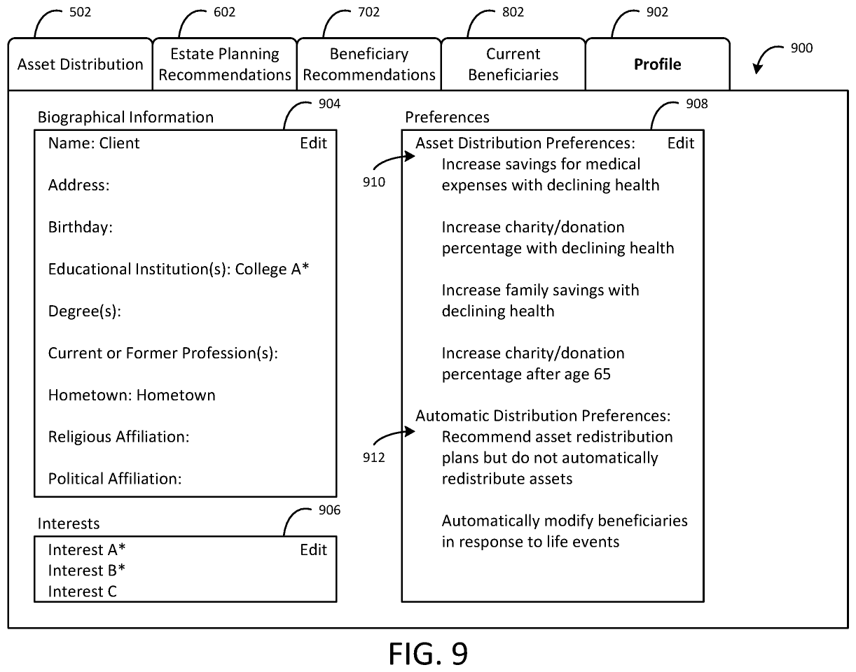

The system manager circuit 250 is configured to gather and store at least some of the biographical information, interests information, assets information, beneficiaries information, asset distribution preferences, etc. for the client at the time the client requests the estate planning prediction and distribution services (e.g., at the time the client signs up for the services through the financial institution associated with the financial institution computing system 102). In some embodiments, the system manager circuit 250 is configured to gather at least some of the information or gather additional information at some time after the client requests the estate planning prediction and distribution services. In one example, in response to the beneficiary recommendation circuit 278 determining that it should recommend potential beneficiaries to the client, as described in further detail below, the system manager circuit 250 notifies the client (e.g., via the notification circuit 254) to provide or update biographical information and/or interests information to the computing system 102. In another example, the client has access to a profile page for the client hosted by the computing system 102 (e.g., as shown in FIG. 9, discussed in further detail below) through which the client is able to update biographical information, interests information, asset distribution preferences, and so on, at any time.

The notification circuit 254 is structured to provide various notifications to the client. Examples of client notifications include requests for biographical and interests information (e.g., as described above with respect to the system manager circuit 250), recommended estate planning activities for the client to complete (e.g., as described below with respect to the estate planning recommendation circuit 274), recommended asset redistribution plans (e.g., as described below with respect to the asset distribution recommendation circuit 276), and recommended potential beneficiaries for the client (e.g., as described below with respect to the beneficiary recommendation circuit 278). Accordingly, as shown in FIG. 2, the notification circuit 254 is communicably and operatively connected to the system manager circuit 250, the estate planning recommendation circuit 274, the asset distribution recommendation circuit 276, and the beneficiary recommendation circuit 278. In various arrangements, the notification circuit 254 is configured to interface with email applications, with text message applications, with the client application 156, and so on, to provide notifications to the client. In one example, the notification circuit 254 sends the client an email with the notification. In a second example, the notification circuit 254 provides the notification via a push notification on the client application 156. In a third example, the notification circuit 254 sends the client a text message with the notification. In a fourth example, the notification circuit 254 displays the notification on a webpage.

Additionally, in some embodiments, the notification circuit 254 is structured to provide various notifications to a submitter. Examples of submitter notifications include requests for information from the submitter, a confirmation of a submission made by the submitter, and messages informing the submitter that a client of the financial institution has selected the submitter as a beneficiary. Accordingly, as shown in FIG. 2, the notification circuit 254 is further communicably and operatively coupled to the potential beneficiary submission circuit 260. In various arrangements and similar to providing notifications to clients, the notification circuit 254 is configured to interface with email applications, with text message applications, with the submitter application 166, and so on, to provide notifications to the submitter.

The health processing circuit 270 is structured to monitor the client's physical and/or mental health. In monitoring the client's health, the health processing circuit 270 is configured to interface with devices, applications, software, internet accounts, etc. (e.g., by using APIs). For instance, in one example, the health processing circuit 270 is able to read information about a client's health stored on a website or application associated with an activity tracker.

In some embodiments, the health processing circuit 270 monitors the client's health via the client health monitoring device 110. In one example, the health processing circuit 270 monitors the client's health through a device configured to recreationally monitor the client's health, such as an activity tracker (e.g., a Fitbit®), a pedometer, a body scale, or an application on a smart watch or smartphone. In another example, the health processing circuit 270 monitors the client's health through a medical device, such as an implantable medical device (e.g., a pacemaker, a defibrillator, or a neurostimulator) or an external medical device (e.g., a glucose monitor, a blood pressure monitor, or a pulse oximeter) that the client gives the health processing circuit 270 permission to access. Provided that the client provides permission, in yet another example, the health processing circuit 270 alternatively or additionally monitors the client's health through e-medical records for the client, a health rating for the client determined by a health professional, information about a hospital stay of the client, etc., provided to the health processing circuit 270 from a computing system (i.e., another embodiment of the client health monitoring device 110, as discussed above with respect to FIG. 1) by a health professional or healthcare facility associated with the client. In one embodiment, the health processing circuit 270 uses the client health monitoring device 110 to monitor the client's health over time to determine whether the client's wellness has an upward trend, a steady trend, or a downward trend.

In other embodiments, the health processing circuit 270, alternatively or additionally, monitors the client's health via the client device 104. In some arrangements, the health processing circuit 270 uses the client application 156 to access and track various functions of the client device 104 and thereby monitor the client's health. The health processing circuit 270 uses the client application 156 to monitor the client's physical health by, for example, detecting when the client calls for help with the client device 104 related to a physical injury, determining whether the client is consistently enlarging the font on the client device 104, determining whether the client is consistently asking an intelligent personal assistant (IPA) of the client device 104 to repeat itself, detecting whether the client is ordering, via the client device 104, more or certain products related to treating physical ailments, or determining via the client device 104 the client's level of physical activity. Furthermore, the health processing circuit 270 uses the client application 156 to monitor the client's mental health by, for example, determining whether the client often re-asks the same question to the IPA or retypes the same question on a web browser of the client device 104 within a short period of time, determining whether the client often misses events or appointments, determining when the social patterns of the client have drastically changed for no apparent reason (e.g., the client rarely leaves the house when the client used to visit certain friends or certain locations regularly), or determining whether the client uses the client device 104 regularly during the night, indicating that the client is having trouble sleeping.

The health processing circuit 270 is further structured to use the monitored health information to determine a health status of the client. In certain embodiments, the health processing circuit 270 is structured to determine whether the client has poor or declining health. In one embodiment, the health processing circuit 270 determines that the client has poor or declining health based on a current state of the client's health. For example, the health processing circuit 270 determines that the client has poor health because the health processing circuit 270 receives a notification from the client monitoring device 110 that the client was admitted to a hospital. As another example, the health processing circuit 270 determines that the client has poor or declining health because the health processing circuit 270 receives a notification from an implantable pacemaker indicating that it has recently been increasingly activated. As a third example, the health processing circuit 270 determines that the client has poor or declining health because the health processing circuit 270 detects, via the client device 104, that the client has recently been ordering braces and pain medicine from online retailers or pharmacies.

In another embodiment, the health processing circuit 270 determines that the client has poor or declining health based on a downward trend of the client's health. As an example, the health processing circuit 270 regularly receives information from an activity tracker regarding the client's health. The health processing circuit 270 analyzes the received health information and determines that, over time, the client has become less active and has an increased resting heart rate. In response, the health processing circuit 270 concludes that the client has declining health. As another example, the health processing circuit 270 determines that the client has declining mental health because the health processing circuit 270 finds, from the client device 104, that the client consistently asks the IPA of the client device 104 the same question within a short period of time and consistently misses appointments despite being reminded of them by the client device 104.

In yet another embodiment, the health processing circuit 270 reevaluates the client's health status on a regular basis. For instance, in one arrangement, the health processing circuit 270 reevaluates the client's health status on an annual basis. The health processing circuit 270 then compares the client's current health status to the client's health status from the previous year, over the previous five years, and so on to determine whether the client has an upward, steady, or downward health trend. As an example, the health processing circuit 270 receives e-medical records for the client from the client's yearly physical. The health processing circuit 270 determines that the e-medical records show that during the past five years, the client's health has been worse at each subsequent annual physical. Because the client's health is trending downward, the health processing circuit 270 accordingly concludes that the client is in declining health.

As shown in FIG. 2, the estate planning prediction system 256 is communicably and operatively coupled to the estate planning recommendation circuit 274 and the asset distribution recommendation circuit 276. Accordingly, the health processing circuit 270 is structured to provide to the estate planning recommendation circuit 274 and the asset distribution recommendation circuit 276 an indication of the determined health status for the client. In some embodiments, the health processing circuit 270 only provides to the estate planning recommendation circuit 274 and the asset distribution recommendation circuit 276 an indication of the client's health status when the health processing circuit 270 determines that the client is in poor or declining health. In other embodiments, the health processing circuit 270 regularly provides (e.g., annually provides) to the estate planning recommendation circuit 274 and the asset distribution recommendation circuit 276 an indication of the client's current or historical health status.

The estate planning recommendation circuit 274 is structured to recommend that the client carry out estate planning activities in response to an indication of poor or declining health from the health processing circuit 270. In making these recommendations, the estate planning recommendation circuit 274 examines biographical information about the client, information about the client's assets, and information about the client's beneficiaries stored in the client database 128. Accordingly, the estate planning recommendation circuit 274 is communicably and operatively coupled to the client database 128. Based on the examined information, the estate planning recommendation circuit 274 provides to the client various recommendations regarding estate planning activities the client should carry out. For example, in one embodiment, the estate planning recommendation circuit 274 recommends that the client write or update various legal documents related to estate planning (e.g., a will, a living hill, a healthcare power of attorney, a financial power of attorney, a trust, or guardianship instructions). In another embodiment, the estate planning recommendation circuit 274, alternatively or additionally, recommends that the client undertake other activities related to estate planning (e.g., obtaining a life insurance policy, obtaining a funeral/burial insurance policy, updating beneficiaries, or redistributing the client's wealth according to his or her current wishes).

In some embodiments, the estate planning recommendation circuit 274 further provides to the client a recommended timeline for carrying out one or more estate planning activities, with a deadline for completing each activity. As an example, a recommended timeline and/or deadlines are based on a likely time that the estate planning recommendation circuit 274 determines the client's estate plan should be finalized by in order to settle end-of-life financial issues. In one embodiment, the estate planning recommendation circuit 274 further provides estate planning attorney recommendations to the client. The estate planning recommendation circuit 274 bases the attorney recommendations on, for example, where the client lives, ratings for nearby estate planning attorneys, and/or the specialties of the nearby estate planning attorneys. Moreover, those of skill in the art will appreciate that, in various embodiments, the estate planning recommendation circuit 274 provides additional recommendations to the client and/or carries out further actions related to estate planning in response to receiving an indication that the client has poor or declining health from the health processing circuit 270.

Once the estate planning recommendation circuit 274 has determined the estate planning recommendations for the client, the estate planning recommendation circuit 274 provides the estate planning recommendations to the notification circuit 254, which the notification circuit 254 then provides to the client. For example, the estate planning recommendation circuit 274 notifies the client, via the notification circuit 254, of the estate planning recommendations by sending the client an email or sending the client a text message. As another example, the estate planning recommendation circuit 274 notifies the client of the recommendations by having a letter sent to the client. In one embodiment, the estate planning recommendation circuit 274 includes the details of the recommendations in the notification sent to the client. In another embodiment, the estate planning recommendation circuit 274 does not include the details of the recommendations in the notification and instead instructs the client to log into an account (e.g., via the client application 156 of the client device 104) provided to the client through the financial institution to view the details.

In addition to, or alternatively from, the estate planning recommendation circuit 274 recommending that the client carry out estate planning activities, the asset distribution recommendation circuit 276 is structured to recommend to the client a redistribution plan for the client's assets, in response to receiving an indication that the client has poor or declining health from the health processing circuit 270. In making the redistribution recommendation, the asset distribution recommendation circuit 276 examines the information about the client's assets stored in the client database 128 and determines a redistribution plan for the assets. Accordingly, the asset distribution recommendation circuit 276 is communicably and operatively coupled to the client database 128.

Additionally, in some embodiments, the asset distribution recommendation circuit 276 determines a redistribution plan for the client's assets based at least partially on the client's asset distribution preferences (e.g., as gathered by the system manager circuit 250) stored in the client database 128. Thus, in response to an indication that the client has poor or declining health, the asset distribution recommendation circuit 276 retrieves the client's stored asset distribution preferences and recommends an asset redistribution plan in line with those preferences. As an example, the client instructs the system manager circuit 250 that, in response to declining health in the client, the computing system 102 should recommend a redistribution of the client's assets in order to save more money for medical expenses, to increase a donation percentage, and to increase savings for family members' education. Accordingly, once the asset distribution recommendation circuit 276 receives an indication from the health processing circuit 270 that the client's health is poor or declining, the asset distribution recommendation circuit 276 recommends a redistribution of the client's assets to save more money for medical expenses, to designate more assets to be donated, and to save more money for family members' education.

In other embodiments, the asset distribution recommendation circuit 276, additionally or alternatively, recommends a redistribution of the client's assets based at least partially on predicted needs, desires, or interests of the client. In one embodiment, the asset distribution recommendation circuit 276 examines the client's history of asset distributions in predicting the client's needs, desires, or interests. In another embodiment, the asset distribution recommendation circuit 276 predicts the client's needs, desires, or interests based at least partially on the client's biographical and/or interests information stored in the client database 128. Moreover, as shown in FIG. 2, the asset distribution recommendation circuit 276 is communicably and operatively coupled to the system manager circuit 250. As such, in yet another embodiment, the asset distribution recommendation circuit 276 gathers additional information about the client via the system manager circuit 250 and bases the needs, desires, or interest predictions on the additional gathered information. In one example, the system manager circuit 250 gathers additional information about the client's age, family, financial health, and so on, which the asset distribution recommendation circuit 276 takes into account when making predictions about the client's needs, desires, or interests. For instance, the asset distribution recommendation circuit 276 predicts that, based on the fact that the client has two minor children and no educational savings dedicated to the children, the client would like to save money for the children's education. Subsequently, in response to receiving an indication that the client has poor or declining health from the health processing circuit 270, the asset distribution recommendation circuit 276 recommends that the client redistribute his or her assets in order to start an educational fund for the client's children.

In further embodiments, the asset distribution recommendation circuit 276 additionally recommends a redistribution of the client's assets based on one or more “redistribution triggers” aside from poor or declining health. When used herein, “redistribution trigger” means the occurrence of an event or the fulfillment of a condition that triggers the asset distribution recommendation circuit 276 to create an asset redistribution plan. Examples of additional redistribution triggers include the client reaching a certain age, the client's financial health being poor or well, potential tax benefits for the client if the client's assets are redistributed, life events (as described in further detail below), and so on. In one embodiment, the client provides the additional triggers to the computing system 102 as part of the client's asset distribution preferences. In another embodiment, the asset distribution recommendation circuit 276 predicts that the client needs or would like a redistribution of his or her assets based on a redistribution trigger aside from poor or declining health.

In various arrangements of the asset distribution recommendation circuit 276, the asset distribution recommendation circuit 276 recommends a plan for redistributing the client's assets and provides a notification of the plan to the client (e.g., by the notification circuit 254). In other arrangements, the asset distribution recommendation circuit 276 automatically redistributes at least some of the client's assets according to the asset distribution plan determined by the asset distribution recommendation circuit 276. In one embodiment, the system manager circuit 250 gathers from the client instructions on whether to automatically redistribute and/or which assets to automatically redistribute as part of the gathered client asset preferences. As an example, the client instructs the computing system 102 to (a) automatically redistribute certain assets and (b) for other assets, to recommend to the client a redistribution plan without automatically redistributing them. The asset distribution recommendation circuit 276 then determines whether to notify the client of the redistribution plan and/or to automatically redistribute the assets based on the automatic redistribution preferences stored for the client in the client database 128.

If the client decides to redistribute the assets and/or if the asset distribution recommendation circuit 276 automatically redistributes the client's assets, the asset distribution recommendation circuit 276 updates information about the client's assets in the client database 128. Additionally, as with the estate planning recommendation circuit 274, when the asset distribution recommendation circuit 276 determines a redistribution plan for the client's assets, the asset distribution recommendation circuit 276 provides a notification regarding the redistribution plan to the notification circuit 254. The notification circuit 254 then provides the notification to the client. For example, the asset distribution recommendation circuit 276 notifies the client of the redistribution plan by sending the client an email or by sending the client a text message. As another example, the asset distribution recommendation circuit 276 notifies the client by having a letter sent to the client. In one embodiment, the asset distribution recommendation circuit 276 includes the details of the redistribution plan in the notification sent to the client. In another embodiment, the asset distribution recommendation circuit 276 does not include the details of the redistribution plan in the notification and instead instructs the client to log into an account (e.g., via the client application 156 of the client device 104) for the client through the financial institution to view the details.

As described above, in response to the health processing circuit 270 determining that the client has poor or declining health, the estate planning recommendation circuit 274 provides estate planning recommendations to the client and/or the asset distribution recommendation circuit 276 provides an asset redistribution plan to the client. Accordingly, in some cases, as a result of the client performing estate planning activities and/or the client's assets being redistributed according to the redistribution plan, a larger percentage of the client's assets are earmarked for donation. In those cases, the beneficiary recommendation circuit 278 is structured to provide one or more recommendations to the client for potential beneficiaries for the increased earmarked donation percentage, as described herein.

In certain embodiments, the beneficiary recommendation circuit 278 is structured to select and recommend potential beneficiaries to the client from one or more potential beneficiaries stored in the potential beneficiaries database 138. As such, the beneficiary recommendation circuit 278 is communicably and operatively coupled to the potential beneficiaries database 138. In various embodiments, the potential beneficiaries included in the potential beneficiaries database 138 consist of charitable organizations, research organizations, political or special interest groups, religious groups, human interest groups, etc. that various individuals may want to select as donation beneficiaries. Each potential beneficiary has characteristics (e.g., traits, goals, causes, locations, or affiliations) associated with it in the potential beneficiaries database 138.

Additionally, in some embodiments, the potential beneficiaries database 138 includes submissions from one or more submitters (e.g., sent to the financial institution computing system by the submitter application 166 of the submitter device 106). As described above, a submitter is an individual, a group, an organization, etc. seeking to raise money by becoming a beneficiary of one or more clients of the financial institution. In some arrangements, the submitter aims to raise money for a particular project, while in other arrangements, the submitter is generally raising money. Each submission includes a written and/or video description of the reasons why, in the submitter's opinion, one or more clients should select the submitter as a beneficiary. For example, in some arrangements, the submission includes a written and/or video description discussing the submitter, the goals of the submitter, a project for which the submitter hopes to receive money, a description of the ways that the submitter and/or the project will be beneficial to society, and so on. In one example, a submitter is an individual, and the submission describes how the submitter hopes to attend college in order to research cures for cancer but that the submitter needs funding in order to attend college. In some submissions, the submitter asks for a specific amount of money in the submission. In other submissions, the submitter generally asks to be selected as a beneficiary without designating a particular amount.

As shown in FIG. 2, the estate planning system 140 includes the potential beneficiary submission circuit 260. The potential beneficiary submission circuit 260 is structured to receive potential beneficiary submissions from submitters. In various examples, the potential beneficiary submission circuit 260 has an associated website, an associated application for a smartphone or tablet computer, or a dedicated portion of a financial institution application for a smartphone or tablet computer, whereby submitters create and provide submissions to the potential beneficiary submission circuit 260 (e.g., by using the submitter application 166 of the submitter device 106). Additionally, in some embodiments, the potential beneficiary submission circuit 260 also interfaces with various platforms not associated with the financial institution (e.g., by using APIs), such as social media, crowdfunding platforms, and so on. Submitters are thereby able to create and submit potential beneficiary submissions to the potential beneficiary submission circuit 260 through the interfacing platforms.

Furthermore, in some embodiments, the potential beneficiary submission circuit 260 also solicits potential beneficiary submissions. As an example, the potential beneficiary submission circuit 260 looks for existing fundraising or crowdfunding campaigns on crowdfunding platforms or blogs. The potential beneficiary submission circuit 260 then contacts the individual(s) or organization(s) running an existing campaign and asks the individual(s) or organization(s) to create and provide a potential beneficiary submission to the financial institution computing system 102. Alternatively, the potential beneficiary submission circuit 260 creates a potential beneficiary submission on the behalf of the individual(s) or organization(s) by, for example, extracting information about the existing campaign from the crowdfunding platform or blog.

Regardless of how it occurs, when a submitter provides a potential beneficiary submission to the financial institution computing system 102, the submission is received by the potential beneficiary submission circuit 260, and the potential beneficiary submission circuit 260 is configured to retrievably store the submission in the potential beneficiaries database 138. Accordingly, as shown in FIG. 2, the potential beneficiary submission circuit 260 is communicably and operatively coupled to the potential beneficiaries database 138. In some embodiments, before storing the submission in the potential beneficiaries database 138, the potential beneficiary submission circuit 260 stores the submission in a temporary memory or database while an administrator reviews the submission to determine if it is an appropriate submission (e.g., to ensure that the submission is directed to an appropriate type of organization or project). Once the administrator approves of the submission, the potential beneficiaries submission circuit 260 then stores the submission in the potential beneficiaries database 138. The potential beneficiary submission circuit 260 further stores characteristics (e.g., traits, goals, causes, locations, or affiliations) associated with the submission, either as entered by the submitter or as extracted from the submission description, in the potential beneficiaries database 138.

Accordingly, as noted above, the beneficiary recommendation circuit 278 is configured to recommend beneficiaries for the client from the potential beneficiaries database 138. To this end, the beneficiary recommendation circuit 278 is structured to retrieve the client's biographical and/or interests information stored in the client database 128 and, as such, is communicably and operatively coupled to the client database 128. If provided by the client, the beneficiary recommendation circuit 278 further retrieves from the client database 128 a rank, priority, importance, etc. that the client has placed on certain biographical and/or interests information for the purposes of determining potential beneficiaries. The beneficiary recommendation circuit 278 then determines the recommended potential beneficiaries for the client by matching the client's biographical and/or interests information, taking into account any prioritizations the client has made, with the characteristics of various potential beneficiaries stored in the potential beneficiaries database 138. The beneficiary recommendation circuit 278 determines the recommended potential beneficiaries to be the potential beneficiaries with the most commonality with the (prioritized) biographical and/or interests information of the client.

As an example, assume the biographical information for a client, stored in the client database 128, includes the client's address, the undergraduate university the client attended, the client's current profession, a list of organizations to which the client belongs, and the client's hometown. For example, the interests for the client, stored in the client database 128, can include finding a cure to cancer, helping youth, tennis, and dogs. Additionally, the client database 128 stores instructions from the client indicating that the most important pieces of biographical and interests information for potential beneficiary matching purposes are the client's undergraduate university, the client's hometown, finding a cure to cancer, and helping youth.

The beneficiary recommendation circuit 278 retrieves this stored information from the client database 128 and searches for the most closely matching beneficiaries in the potential beneficiaries database 138. After searching through the potential beneficiaries database 138, the beneficiary recommendation circuit 278 finds that the characteristics of Organization A, Organization B, and Organization C are most closely aligned with the client's biographical information and interests. Organization A is a dog shelter in the same city as the client lives. Organization B is an organization in the client's hometown dedicated to helping underprivileged youth learn sports such as tennis. Organization C is a research organization affiliated with the client's undergraduate university that aims to find a cure to cancer. Because Organization B matches three pieces of the client's biographical and interests information (the client's hometown, helping youth, and tennis), the beneficiary recommendation circuit 278 prioritizes Organization B as first. Organizations A and C each match with two pieces of the client's biographical information and interests (dogs and the client's address for Organization A; the client's undergraduate university and finding a cure to cancer for Organization C). However, because Organization C matches with the client information the client indicated as most important, the beneficiary recommendation circuit 278 prioritizes Organization C over Organization A. Thus, the beneficiary recommendation circuit 278 presents to the client Organization B as its top choice for the client, with Organization C and Organization A as its second and third choices, respectively, for the client.

However, in other embodiments, the beneficiary recommendation circuit 278, additionally or alternatively, is structured to recommend relatives for the client as potential beneficiaries. In one embodiment, the beneficiary recommendation circuit 278 determines one or more relatives to recommend to the client as potential beneficiaries based on biographical information about the client and beneficiary information for the client's assets stored in the client database 128. As an example, the beneficiary recommendation circuit 278 determines from a client's biographical and beneficiaries information that the client has a niece and a nephew who are currently not beneficiaries of the client and accordingly recommends that the client add the niece and nephew as beneficiaries.

In another embodiment, the beneficiary recommendation circuit 278 additionally determines one or more relatives to recommend to the client as potential beneficiaries based on commonalities between the interests and/or biographical information of the one or more relatives (i.e., “characteristics” of the relatives) and the client's interests and/or biographical information stored in the client database 128. For example, the beneficiary recommendation circuit 278, via the system manager circuit 250, solicits biographical and/or interests information from various relatives of the client. As another example, the beneficiary recommendation circuit 278, via the system manager circuit 250, gathers information about various relatives of the client from social media, blog postings, conducting online searches, etc. (e.g., by using APIs). The beneficiary recommendation circuit 278 then recommends one or more relatives as potential beneficiaries of the client by matching characteristics of one or more relatives with the biographical and/or interests information of the client, similar to the process described above with respect to using the potential beneficiaries database 138 to determine potential beneficiaries. As an illustration, the beneficiary recommendation circuit 278 determines from social media, via the system manager circuit 250, that the client has a great-niece who is attending the same university that the client attended for her undergraduate degree. The computing system 102 accordingly recommends the great-niece to the client as a potential beneficiary

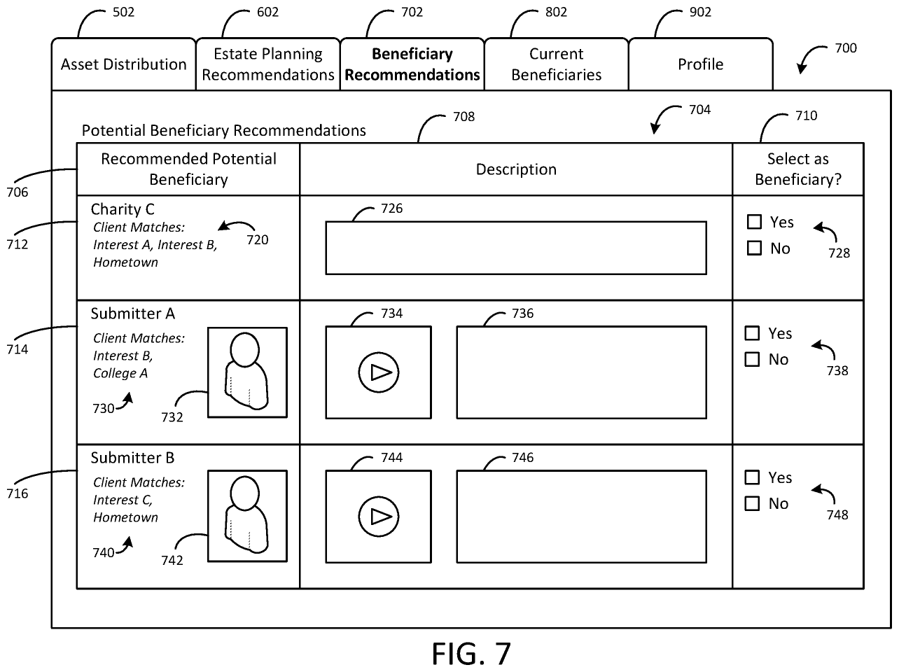

In some embodiments, the beneficiary recommendation circuit 278 simply recommends to the client (e.g., by the notification circuit 254) the potential beneficiaries the beneficiary recommendation circuit 278 determines to be optimally matched with the client's biographical information and/or interests. For example, in one embodiment, the beneficiary recommendation circuit 278 presents a list of potential beneficiaries to the client, with the most optimally matched potential beneficiaries listed first (e.g., as shown in FIG. 7, as described in further detail below). If any of the potential beneficiaries are submitters, the beneficiary recommendation circuit 278 further presents the associated submission to the client so that the client may peruse the written and/or video description of the submission. In some arrangements, the beneficiary recommendation circuit 278 also provides to the client contact information for each recommended potential beneficiary. The client is then able choose from the list of recommended potential beneficiaries which, if any, he or she would like to set as actual beneficiaries and how much money or how much of an asset's percentage he or she would like to provide to each selected beneficiary (e.g., the client can choose to provide the full amount asked for in a submission, or part of the amount asked for in the submission). In another embodiment, the beneficiary recommendation circuit 278 further allows the client to independently peruse the potential beneficiaries database 138 to find beneficiaries.

In other embodiments, the beneficiary recommendation circuit 278 automatically sets one or more recommended potential beneficiaries as actual beneficiaries of the client's assets. For example, the beneficiary recommendation circuit 278 automatically selects the top two most optimally matched potential beneficiaries as actual beneficiaries of an increased donation percentage of the client's assets. In one embodiment, the beneficiary recommendation circuit 278 determines whether to automatically set one or more recommended beneficiaries as actual beneficiaries based on distribution preferences stored for the client in the client database 128. Using the previous example, the beneficiary recommendation circuit 278 sets the top two most optimally matched beneficiaries as actual beneficiaries because distribution preferences for the client stored in the client database 128 indicate that, when a redistribution of the client's assets provides for an increased donation percentage, the financial institution computing system 102 should split the increased donation percentage evenly between the two most optimally matched potential beneficiaries determined for the client. As another example, distribution preferences for a client stored in the client database 128 include instructions to automatically redistribute a portion of an increased donation percentage, determined as part of an asset redistribution plan, to existing designated beneficiaries. The instructions further indicate that the computing system 102 should redistribute the remaining portion of the increased donation percentage to the three potential beneficiaries that the computing system 102 finds most closely match the client's biographical information and interests. The beneficiary recommendation circuit 278 then acts according to these instructions after determining a redistribution plan for the client's assets that includes an increased donation percentage.

If the client selects one or more new beneficiaries and/or the beneficiary recommendation circuit 278 automatically selects one or more recommended potential beneficiaries as actual beneficiaries, the beneficiary recommendation circuit 278 updates the client's beneficiary information in the client database 128. Additionally, when the beneficiary recommendation circuit 278 determines recommended potential beneficiaries for the client, the beneficiary recommendation circuit 278 provides a notification regarding the recommendations to the notification circuit 254. The notification circuit 254 then provides the notification to the client. For example, the beneficiary recommendation circuit 278 sends the client, via the notification circuit 254, an email or a text message. As another example, the beneficiary recommendation circuit 278 has a letter sent to the client. In one embodiment, the beneficiary recommendation circuit 278 does not include the details of the recommendations in the notification and instead instructs the client to log into an account (e.g., via the client application 156 of the client device 104) for the client through the financial institution to view the details of the recommended potential beneficiaries. In another embodiment, the notification circuit 254 includes the notification regarding the potential beneficiaries recommendations from the beneficiary recommendation circuit 278 with one or more notifications regarding estate planning recommendations made by the estate planning recommendation circuit 274 and/or asset distribution recommendations made by the asset distribution recommendation circuit 276.

In some embodiments, when the client or the beneficiary recommendation circuit 278 sets one or more potential beneficiaries as actual beneficiaries, the beneficiary recommendation circuit 278 also provides a notification to the notification circuit 254, which the notification circuit 254 then provides to the one or more potential beneficiaries, informing them that they have been selected as actual beneficiaries. In one embodiment, the notification circuit 254 provides to the newly selected one or more beneficiaries contact information for the client that selected them. In another embodiment, the notification circuit 254 provides to the newly selected one or more beneficiaries a notification simply stating that they have been selected and instructing them to log into an account through the financial institution (e.g., via the submitter application 166 of the submitter device 106) to view more details.

As shown in FIG. 2, the estate planning system 140 further includes the event determination circuit 272. The event determination circuit 272 is structured to determine whether a life event has occurred for a client. As used herein, a life event is a birth (e.g., the birth of a child, the birth of a grandchild, or the birth of a niece or nephew), a marriage (e.g., the client marries or a child of the client marries), a divorce (e.g., the client divorces, a child of the client divorces, or the client's parents' divorce), a death (e.g., the death of a parent of the client, the death of a grandparent of the client, the death of the client's spouse, or the death of a child of the client), and so on, in the life of the client. In some embodiments, the event determination circuit 272 is structured to determine that a life event has occurred based on client input, submitted either directly to the estate planning system 140 or to the financial institution more generally. For example, the client logs into an online account associated with the financial institution computing system 102 and updates biographical information about the client to include the life event (e.g., to include a newly born child or a new spouse). As another example, the client updates an account the client holds with the financial institution associated with the financial institution computing system 102 (e.g., a checking account or a savings account) to include or re remove an individual related to a life event for the client (e.g., to include a new spouse). The event determination circuit 272 accordingly determines that a life event has occurred for the client based on the updated account information.

In other embodiments, the event determination circuit 272 is structured to determine, alternatively or additionally, that a life event for the client has occurred based on other sources. For example, in one embodiment, the event determination circuit 272 is structured to retrieve biographical information for the client stored in the client database 128. Accordingly, the event determination circuit 272, as shown in FIG. 2, is communicably and operatively coupled to the client database 128. The event determination circuit 272 then uses the client's biographical information to monitor for life events of the client, such as through social media, by searching public records, based on a search history or buying history retrieved from the client device 104 through the client application 156 (e.g., if the client has been buying diapers through online retailers, the event determination circuit 272 determines that the client has recently had or will have a baby), and so on.

In still other embodiments, the event determination circuit 272 interfaces with assets or accounts held by one or more relatives of the client (e.g., with each relative's permission) and determines that a life event has occurred for the client based on information entered by the one or more relatives relating to their assets or accounts. In one example, the client and the client's sister both hold accounts at the financial institution, and the client's sister gives the computing system 102 permission to access information from her account as it relates to the client. The client's sister later updates her account information to include a newly born son as a beneficiary. This information is received by the event determination circuit 272, which accordingly determines that a life event has occurred for the client (i.e., the client has a new nephew).

Once the event determination circuit 272 has determined that a life event has occurred for the client, the event determination circuit 272 provides information about the life event to the beneficiary recommendation circuit 278. In some embodiments, the event determination circuit 272 additionally provides the life event to the asset distribution recommendation circuit 276. Accordingly, as shown in FIG. 2, the event determination circuit 272 is communicably and operatively coupled to the asset distribution recommendation circuit 276 and the beneficiary recommendation circuit 278.

In addition to the functions described for the beneficiary recommendation circuit 278 above, upon receipt of the life event information, the beneficiary recommendation circuit 278 is further structured to (a) determine a potential beneficiary for the client (e.g., if the life event is the birth of a grandchild, the beneficiary recommendation circuit 278 determines the grandchild to be a potential beneficiary), or (b) to remove an existing beneficiary (e.g., if the life event is a divorce of the client, the beneficiary recommendation circuit 278 determines that the ex-spouse should be removed as a beneficiary of the client). In some embodiments, the beneficiary recommendation circuit 278 then provides to the client, via the notification circuit 254, a notification that the client should update his or her beneficiaries. In other embodiments, the beneficiary recommendation circuit 278, additionally or alternatively, automatically updates one or more client assets to include a new beneficiary and/or to remove an existing beneficiary based on the life event.

For example, the beneficiary recommendation circuit 278 retrieves information about the client's assets and current beneficiaries from the client database 128. Based on the retrieved information, the beneficiary recommendation circuit 278 determines where to add a new beneficiary as a result of the life event and/or where to remove an existing beneficiary as a result of the life event. The beneficiary recommendation circuit 278 then examines the client database 128 to determine whether the client has given the computing system 102 permission to automatically modify the client's beneficiaries in response to life events. If the client has given permission, the beneficiary recommendation circuit 278 then automatically adds and/or removes these beneficiaries and updates the client database 128 accordingly. If the client has not given permission, the beneficiary recommendation circuit 278 simply provides a notification to the client, via the notification circuit 254, to update the client's beneficiaries. As a more specific example, the beneficiary recommendation circuit 278 receives an indication from the event determination circuit 272 that the client has had a child. In response, the beneficiary recommendation circuit 278 determines that the client should add the new child as a beneficiary of the client's 401k and either notifies the client to add the child as a beneficiary or automatically adds the child as a beneficiary.

Furthermore, in addition to the functions for the asset distribution recommendation circuit 276 above, upon receipt of the life event information from the event determination circuit 272 the asset distribution recommendation circuit 276 is further structured to recommend that the client redistribute, and/or is structured to automatically redistribute, the client's assets. In doing so, the asset distribution recommendation circuit 276 is configured to form an asset redistribution recommendation plan as previously discussed above. Using the previous example, the asset distribution recommendation circuit 276 recommends that the client redistribute his or her assets to save more money for the new child's education, or the asset distribution recommendation circuit 276 automatically redistributes the client's assets to provide for an education fund for the client's new child.

If updating the client's beneficiaries in response to a life event and/or a recommended redistribution plan based on a life event provide for a larger donation percentage (e.g., the client's spouse dies or the client gets divorced, leaving the client with funds previously allotted to the deceased or ex-spouse needing to be redistributed), the beneficiary recommendation circuit 278 is also configured to recommend potential beneficiaries to the client. In various embodiments, the beneficiary recommendation circuit 278 recommends potential beneficiaries to the client in the same manner as discussed above.

As an example of the above-described estate planning in response to life events, the client provides to the system manager circuit 250 an instruction to automatically add new beneficiaries based on life events and to automatically redistribute the client's assets in response to a life event that is a birth of a child, which the system manager circuit 250 retrievably stores in the client database 128. The event determination circuit 272 subsequently determines that the client has had a new child based on the client's and the client's spouse's social media postings. The event determination circuit 272 provides an indication of the life event to the asset distribution recommendation circuit 276 and to the beneficiary recommendation circuit 278. In response, the beneficiary recommendation circuit 278 determines that the new child should be added as a beneficiary. The beneficiary recommendation circuit 278 retrieves information about the client's assets from the client database 128 and automatically updates the client's assets to list the new child as a beneficiary. The beneficiary recommendation circuit 278 then updates the client database 128 to include the new child. The asset distribution recommendation circuit 276 also retrieves information about the client's assets from the client database 128. Based on the client's preference for automatic redistribution in response to the birth of a child, and a prediction that the child will need an education fund, the asset distribution recommendation circuit 276 automatically redistributes the client's assets to increase the amount of assets directed to an educational fund for the client's children. The asset distribution recommendation circuit 276 and the beneficiary recommendation circuit 278 then send a message to the client, via the notification circuit 254, informing the client of the new child being included as a beneficiary of the client's assets and of the asset redistribution.

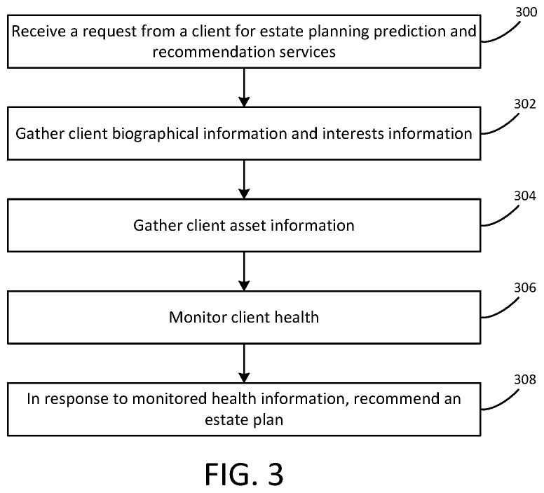

Referring now to FIG. 3, a method of providing estate planning predictions and recommendations by the financial institution computing system 102 is shown, according to an example embodiment. A request from a client for estate planning prediction and recommendation services is received at 300. In some arrangements, the client is a new client of the financial institution associated with the financial institution computing system 102, while in other arrangements, the client is an existing client of the financial institution provided with additional estate planning services by the computing system 102.

Client biographical and interests information is gathered at 302. In various embodiments, the financial institution computing system 102 gathers the biographical and interests information from the client directly, from the financial institution associated with the computing system 102, and/or from third parties. As an example, the financial institution computing system 102 gathers biographical and interests information about the client from social media, by performing internet searches on the client, and/or from other organizations or companies associated with the client.

Client asset information is gathered at 304. In one embodiment, the financial institution computing system 102 asks the client directly for information about the client's assets. In another embodiment, the computing system 102 searches public records to find client assets. In some arrangements, information about the client's current or intended beneficiaries and/or the client's distribution preferences for the client's assets are also gathered at 304. Furthermore, in some arrangements, the client provides the computing system 102 permission to monitor and/or automatically redistribute the client's assets and/or reassign the client's beneficiaries at 304.

The client's health is monitored at 306. Accordingly, the computing system 102 is able to interface with devices, applications, software, internet accounts, etc., as part of monitoring the client's health. In various arrangements, monitoring the client's health includes monitoring the client's physical health, as well as monitoring the client's mental health. In some embodiments, the financial institution computing system 102 monitors the client's health through the client health monitoring device 110 (e.g., an activity tracker, a pedometer, a body scale, a smart watch, an implantable or external medical device, or a computing system used by a health professional or healthcare facility). In other embodiments, the financial institution computing system 102 monitors the health of the client through the client device 104 (e.g., through the client application 156 on the client device 104). The financial institution computing system 102 then uses the monitored client health information to determine a health status for the client. In various arrangements, the computing system 102 is further configured to determine whether the client has poor or declining health, based on a current health status of the client and/or based on a downward trend of the client's health statuses over time.

In response to the monitored health information, an estate plan is recommended at 308. In certain embodiments, the financial institution computing system 102 recommends an estate plan based on the client having poor or declining health (e.g., in response to the annual evaluation of the client's health showing that the client's health has declined from the previous year). As discussed above with respect to FIG. 2, the estate plan recommended by the computing system 102 includes notifying the client to carry out recommended estate planning activities. For example, the recommended estate plan includes notifying the client to write or update various legal documents related to estate planning, such as a will, a living will, or a healthcare power of attorney, and/or the recommended estate plan includes notifying the client to undertake other activities related to estate planning, such as obtaining a life insurance policy, updating beneficiaries for an existing asset, or redistributing the client's wealth according to the client's current wishes. In one embodiment, the financial institution computing system 102 provides a timeline for carrying out various estate planning activities, including a proposed deadline for each activity. In another embodiment, the computing system 102 further provides estate planning attorney recommendations for the client.

Alternatively, or additionally, the estate plan recommended by the financial institution computing system 102 includes a redistribution plan for the client's assets. In one embodiment, the financial institution computing system 102 recommends the redistribution of the client's assets based on asset distribution preferences the client has previously provided to the computing system 102. In another embodiment, the financial institution computing system 102, additionally or alternatively, recommends the redistribution of the client's assets based on predicted needs, desires, or interests of the client. While in some arrangements, the financial institution computing system 102 simply recommends a plan for redistributing the client's assets, in other arrangements, the financial institution computing system 102 automatically redistributes some or all of the client's assets according to the recommended asset redistribution plan. Accordingly, in certain arrangements, the computing system 102 asks the client whether the client would like the computing system 102 to automatically redistribute the client's assets based on a recommended redistribution plan or to just recommend the plan to the client. The computing system 102 then acts according to the client's preferences.

Furthermore, in response to an asset redistribution as a result of recommended estate planning activities and/or an asset redistribution plan providing for a higher donation percentage of the client's assets, the financial institution computing system 102 recommends potential beneficiaries for the client, based on the client biographical and/or interests information gathered in step 302. In various embodiments, the computing system 102 recommends potential beneficiaries for the client from the various individuals and organizations stored in the potential beneficiaries database 138 (e.g., including submitters that have sent funding submissions to the potential beneficiaries database 138) and/or by finding relatives of the client that the client has not already set as beneficiaries. Furthermore, in various embodiments, the computing system 102 recommends potential beneficiaries based on commonalities between characteristics of the potential beneficiaries and the client biographical and/or interests information.

In some arrangements, in response to a redistribution of the client's assets providing for a higher donation percentage, the financial institution computing system 102 simply recommends potential beneficiaries to the client. In other arrangements, the financial institution computing system 102 automatically sets one or more recommended potential beneficiaries as actual beneficiaries of the client's increased donation percentage (e.g., according to which of the potential beneficiaries has the most commonality with the client's prioritized biographical information and/or interests). In still other embodiments, the computing system 102 asks the client whether the client would like the computing system 102 to automatically set one or more recommended potential beneficiaries as actual beneficiaries for the client's assets and acts according to the client's preferences.

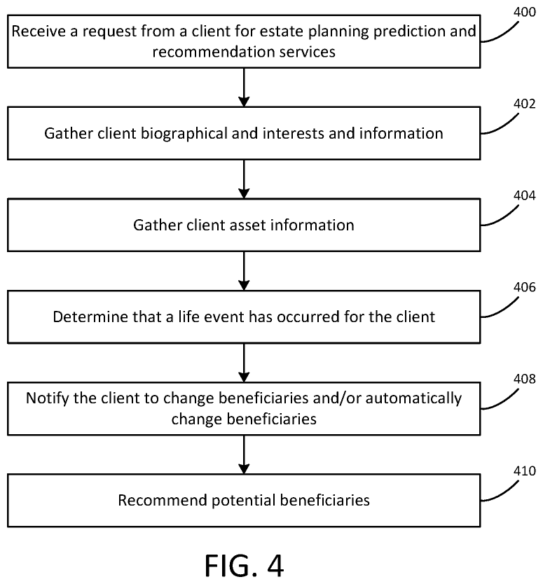

Referring now to FIG. 4, a method of providing beneficiary recommendations, by the financial institution computing system 102, based on life events of the client is shown, according to an example embodiment. A request from a client for estate planning prediction and recommendation services is received at 400. Client biographical and interests information is gathered at 402. Client asset information is gathered at 404. The financial institution computing system 102 accordingly performs steps 400, 402, and 404 similarly to steps 300, 302, and 304 described above with respect to FIG. 3.

That a life event has occurred for the client is determined at 406. As discussed above, a life event is a birth, a marriage, a divorce, a death, and so on, in the life of the client. In one embodiment, the financial institution computing system 102 determines that a life event has occurred for the client based on client input (e.g., the client adds a new spouse to a checking account held through the financial institution of the financial institution computing system 102). In another embodiment, the financial institution computing system 102 determines that a life event has occurred for the client by monitoring social media, public records, and so on. In yet another embodiment, the financial institution computing system 102 determines that a life event has occurred for the client by interfacing with assets or accounts held by family members of the client (e.g., the client's son adds a new child as a beneficiary of an account held through the financial institution, thereby notifying the computing system 102 that a grandchild of the client has been born).

The client is notified to change the client's beneficiaries and/or beneficiaries are automatically changed at 408. The financial institution computing system 102 determines, based on the life event from step 406, that the client should add a new beneficiary and/or remove an existing beneficiary. In addition to notifying the client to change beneficiaries and/or automatically changing beneficiaries, in some embodiments, the financial institution computing system 102 also recommends that the client redistribute, or automatically redistributes, the client's assets in response to the life event of the client.

Potential beneficiaries for the client are recommended at 410. In various embodiments, in response to beneficiary changes based on client life events providing more assets for donations, the financial institution computing system 102 recommends potential beneficiaries for the assets newly earmarked for donation. The financial institution computing system 102 recommends potential beneficiaries for the client similar to the process described above with respect to step 308 of FIG. 3.

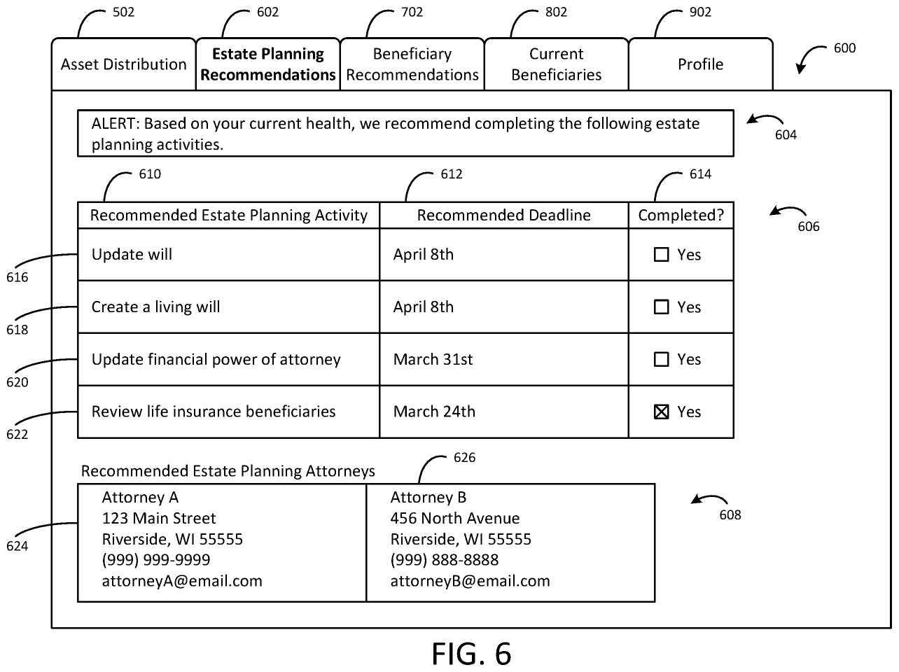

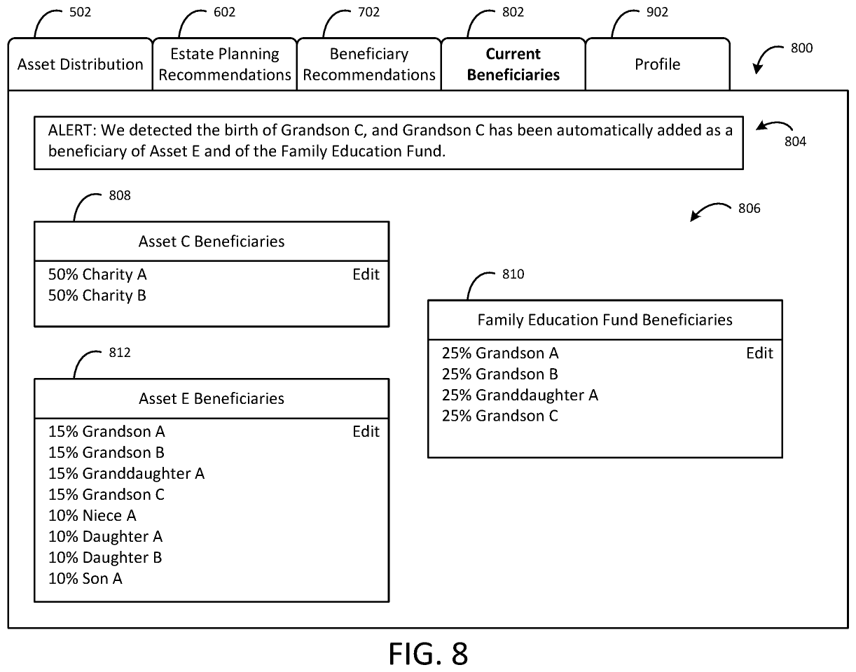

FIGS. 5-9 illustrate example graphical user interfaces presented to a client as part of the estate planning prediction and recommendation services offered by the financial institution computing system 102. FIG. 5 depicts an example screenshot of an asset distribution page 500 that a client observes when using the estate planning prediction and recommendation system 100 through, for example, a user device such as the client device 104. FIG. 6 depicts an example screenshot of an estate planning recommendations page 600 that a client observes when using the system 100. FIG. 7 depicts an example screenshot of a beneficiary recommendations page 700 that a client observe when using the system 100. FIG. 8 depicts a current beneficiaries page 800 and FIG. 9 depicts a profile page 900 that a client observes when using the system 100. In some embodiments, the asset distribution page 500, the estate planning recommendations page 600, the beneficiary recommendations page 700, the current beneficiaries page 800, and the profile page 900 are provided as webpages on a user device, such as through the client application 156 of the client device 104. In other embodiments, the pages 500, 600, 700, 800, and 900 are embodied as an application running on a user device, such as through the client application 156 of the client device 104. As illustrated in FIGS. 5-9, each page 500, 600, 700, 800, and 900 includes five tabs: an asset distribution tab 502, an estate planning recommendations tab 602, a beneficiary recommendations tab 702, a current beneficiaries tab 802, and a profile tab 902. The tabs 502, 602, 702, 802, and 902 indicate which of the associated pages 500, 600, 700, 800, and 900 the client is viewing.

As shown in FIG. 5, the asset distribution tab 502 is bolded, indicating that the client is viewing the asset distribution page 500. The asset distribution page 500 includes an assets section 504 that presents information about the current and/or recommended distribution of the client's assets, as well as an alert section 506 that provides alerts or messages to the client. In the example of FIG. 5, the alert section 506 indicates to the client that, based on the current state of the client's health, the financial institution computing system 102 recommends that the client redistribute his or her assets. In various embodiments, the financial institution computing system 102 determines whether to create an asset redistribution plan and creates the asset redistribution plan as described above with respect to FIGS. 2 and 3. Accordingly, the assets section 504 includes a column 508 listing the client's assets, a column 510 listing the current distribution for each of the assets, and a column 512 listing a recommended distribution for each of the assets. The assets section 504 further includes a column 514 that allows the client to select whether he or she accepts or rejects the recommended distribution for each of the client's assets.

As shown in FIG. 5, rows 516, 518, 520, 522, and 524 of the assets section 504 list the client's assets. Examples of client assets shown in the assets section 504 include checking accounts, savings accounts, interest from savings accounts, stock dividends, rental property income, life insurance, and a 401k. Row 516 lists Asset A, which is currently 100% distributed into a general savings account and which the financial institution computing system 102 recommends that the client distribute 50% into the general savings account and 50% into a medical expenses savings account. Row 518 lists Asset B. Asset B is also currently 100% distributed into the general savings account, but the financial institution computing system 102 recommends that the client distribute 50% of Asset B into the general savings account and 25% into the medical expenses savings account. The financial institution computing system 102 further recommends that the client earmark 25% of Asset B for donations. Row 518 also includes a button 526 for the client to press to select a beneficiary for the 25% donation percentage. In some embodiments, pressing the button 526 directs the client to the beneficiary recommendations page 700, which is discussed in further detail below. Row 520 lists Asset C, which is currently 50% distributed to Charity A and 50% distributed to Charity B and for which the financial institution computing system 102 recommends no changes. Row 522 lists Asset D. Asset D is currently 75% distributed to a family education fund and 25% distributed to general savings, but the financial institution computing system 102 recommends that the client distribute 100% of Asset D to the family education fund. Finally, row 524 lists Asset E, which is currently 100% distributed to listed relative beneficiaries (e.g., the beneficiaries designated for Asset E on the current beneficiaries page 800) and for which the financial institution computing system 102 recommends no changes.

Rows 516, 518, and 522 further include a section in the acceptance/rejection column 514 in which the client may select “Yes”, indicating that the client accepts the recommendations of the financial institution computing system 102 for that asset, or in which the client may select “No”, indicating that the client rejects the recommendations of the financial institution computing system 102 for that asset. However, those of skill in the art will recognize that FIG. 5 is an example embodiment of the asset distribution page 500 and that in other embodiments, the financial institution computing system 102 automatically redistributes some or all of the client's assets according to the determined asset redistribution plan, as described above with respect to FIGS. 2 and 3.

Referring now to FIG. 6, the estate planning recommendations tab 602 is bolded, indicating that the client is viewing the estate planning recommendations page 600. As shown in FIG. 6, the estate planning recommendations page 600 includes an alert section 604 that provides alerts or messages to the client, an estate planning activity section 606, and a recommended attorney section 608. In the example of FIG. 6, the alert section 604 indicates to the client that, based on the client's current health, the financial institution computing system 102 recommends that the client complete the estate planning activities shown in the estate planning activity section 606. As shown, the estate planning activity section 606 includes a column 610 listing the recommended estate planning activities, a column 612 listing a recommended deadline for each recommended estate planning activity, and a column 614 asking the client whether he or she has completed each estate planning activity. The estate planning activity section 604 further includes rows 616, 618, 620, and 622 that list the recommended estate planning activities. Row 616 lists the activity of updating the client's will with a recommended deadline of April 8th. Row 618 lists the activity of creating a living will, also with a recommended deadline of April 8th. Row 620 lists the activity of updating a financial power of attorney with a recommended deadline of March 31st, and row 622 lists the activity of reviewing life insurance beneficiaries with a recommended deadline of March 24th. As discussed above with respect to the estate planning recommendation circuit 274 of FIG. 2, the computing system 102 determines the recommended estate planning activities based on biographical information about the client, information about the client's assets, and/or information about the client's beneficiaries (e.g., stored in the client database 128). For example, for the recommendation of row 616, the computing system 102 is recommending that the client update the client's will because the computing system 102 has determined that the client has a will, based on information in the client database 128, that the client has not updated in some time.

As shown, the client has also indicated in the intersection of the completion column 614 and row 622 that the client has completed the activity of reviewing his or her life insurance beneficiaries. In some embodiments, the client manually submits to the computing system 102 that the client has completed the estate planning activity. In other embodiments, the computing system 102 determines that the client has completed the activity by monitoring the client's assets, monitoring information in public records, and so on, and automatically updates the completion column 614 once the computing system 102 determines that estate planning activities have been completed.

In the example of FIG. 6, the attorney recommendation section 608 includes subsections 624 and 626 with information about attorneys recommended for the client by the computing system 102. Subsection 624 includes the address, phone number, and email for Attorney A, and subsection 626 includes the address, phone number, and email for Attorney B. As discussed above, in various embodiments, the computing system 102 recommends estate planning attorneys for the client based on the client's location, online recommendations and/or satisfaction ratings for the attorney, the attorney's specialty and how the specialty matches with the recommended estate planning activities listed in column 610, and so on. In some embodiments, the client is further able to contact the one or more recommended attorneys through the attorney recommendation section 608 (e.g., by pressing a button that puts the client in contact with the attorney).

Referring now to FIG. 7, the beneficiary recommendations tab 702 is bolded, indicating that the client is viewing the beneficiary recommendations page 700. As shown in FIG. 7, the beneficiary recommendations page 700 includes a section 704 indicating the recommendations for potential beneficiaries for the client as determined by the financial institution computing system 102 (e.g., as described above with respect to the beneficiary recommendation circuit 258 of FIG. 2). As shown, the potential beneficiaries recommendation section includes a column 706 listing the recommended potential beneficiaries, a column 708 listing a description for each recommended potential beneficiary, and a column 710 allowing the client to select the potential beneficiary as an actual beneficiary or reject the potential beneficiary.

Rows 712, 714, and 716 list example recommended potential beneficiaries for the client. To begin with, row 712 lists Charity C as a recommended potential beneficiary. Row 712 includes a portion 720 indicating how the characteristics of Charity C (e.g., its goals, location, or affiliations) match with the biographical information and/or interests of the client. In the case of Charity C, Charity C matches with the Interest A, the Interest B, and the Hometown of the client (e.g., as shown on the profile page 900). Row 712 also includes a written description 726 of Charity C.

Rows 714 and 716 list Submitter A and Submitter B, respectively. Accordingly, rows 714 and 716 are populated with information taken from the submissions (as described above with respect to the potential beneficiary submission circuit 260 of FIG. 2) from Submitter A and Submitter B, respectively. Row 714 includes a portion 730 indicating how the characteristics of Submitter A and/or the characteristics of a project that Submitter A is raising funds for match with the client's interests. Similarly, row 716 includes a portion 740 indicating how the characteristics of Submitter B and/or the characteristics of a project that Submitter B is raising funds for match with the client's interests. As shown in portions 730 and 740, Submitter A matches with the Interest B and the College A associated with the client (e.g., as shown on the profile page 900), and Submitter B matches with the Interest C and the Hometown associated with the client (e.g., as shown on the profile page 900). Rows 714 and 716 each further include a picture 732 and a picture 742, respectively, that Submitters A and B included with their respective submissions. For example, in various arrangements, the picture 732 is a profile picture for Submitter A, a picture of Submitter A's accomplishments, a picture associated with the goals of Submitter A or a project Submitter A would like to accomplish, and so on.

Rows 714 and 716 further include descriptions of Submitters A and B, respectively, taken from the submissions of Submitters A and B. As shown in FIG. 7, row 714 includes a video description 734 and a written description 736. Row 716 similarly includes a video description 744 and a written description 746. For example, in various arrangements, the video description 744 and the written description 746 describe Submitter B, the goals of Submitter B, a project for which Submitter B is raising the funds, and so on.

As shown in FIG. 7, rows 712, 714, and 716 each include a portion (i.e., shown as portion 728 of row 712, portion 738 of row 714, and portion 748 of row 716) in which the client may select “Yes” or “No” for each of Charity C, Submitter A, and Submitter B. The client selects “Yes” to indicate that the client wants to select the recommended potential beneficiary as a beneficiary, or the client selects “No” if the client does not want to select the recommended potential beneficiary as a beneficiary. In one embodiment, for example, if the client selects “Yes” in portion 728 (i.e., selects “Yes” for Charity C), the client is redirected to a different page asking the client to enter in an amount or percentage to provide to Charity C, for which assets the client would like to set Charity C as a beneficiary, etc. If, however, the client instead selects “No” in portion 728 (i.e., selects “No” for Charity C), the financial institution computing system 102 replaces row 712 and Charity C with a different potential beneficiary that shares commonalities with the client.

In some embodiments, when the client visits the beneficiary recommendations page 700, the client is always presented with potential beneficiary recommendations. In other embodiments, when the client visits the beneficiary recommendations page 700, the client is only presented with potential beneficiary recommendations if the financial institution computing system 102 has created an asset redistribution plan for the client's assets with an increased donation percentage (e.g., as the computing system 102 recommends for Asset B as shown on the asset distribution page 500 in row 518).

Referring now to FIG. 8, the current beneficiaries tab 802 is bolded, indicating that the client is viewing the current beneficiaries page 800. As shown, the current beneficiaries page 800 includes a section 804 that provides alerts or messages to the client. In the example of FIG. 7, the alert section 804 indicates to the client that the financial institution computing system 102 has detected the birth of Grandson C and automatically added Grandson C as a beneficiary of Asset E and the client's family education fund. The current beneficiaries page 800 also includes a section 806 indicating the current beneficiaries of the client's various assets. The current beneficiaries section 806 includes a portion 808 indicating the current beneficiaries of Asset C, a portion 810 indicating the current beneficiaries of the client's family education fund, and a portion 812 indicating the current beneficiaries of Asset E.

As shown in FIG. 8, the Asset C beneficiaries portion 808 lists the current beneficiaries of Asset C as Charity A (50% of Asset C) and Charity B (50% of Asset C). This matches the listing for Asset C in row 816 of the asset distribution page 800. The family education fund beneficiaries portion 810 list the current beneficiaries as Grandson A (25% of the fund), Grandson B (25% of the fund), Granddaughter A (25% of the fund), and Grandson C (25% of the fund). The Asset E beneficiaries portion 812 lists the current beneficiaries of Asset E as Grandson A (15% of Asset E), Grandson B (15% of Asset E), Granddaughter A (15% of Asset E), Grandson C (15% of Asset E), Niece A (10% of Asset E), Daughter A (10% of Asset E), Daughter B (10% of Asset E), and Son A (10% of Asset E).

As indicated in the alert section 804, the financial institution computing system 102 automatically added Grandson C to the list of family education fund beneficiaries 810 and to the Asset E beneficiaries 812. As shown in FIG. 8, the computing system 102 also determined a percentage to distribute to Grandson C. In some embodiments, the computing system 102 modifies the beneficiaries of a client's assets when a life event for the client occurs (e.g., in FIG. 8, the birth of Grandson C) based on preset rules submitted by the client. For example, in one arrangement, the client for FIG. 8 has entered preset rules for the family education fund beneficiaries portion 810 and the Asset E beneficiaries portion 812 (e.g., using the “Edit” button shown in each of portion 810 and portion 812), indicating to the computing system 102 to add any newly born grandchildren as beneficiaries. For the family education fund beneficiaries portion 810, the client has instructed the computing system 102 to split the fund equally between all grandchildren. For the Asset E beneficiaries portion 812, the client has instructed the computing system 102 to split 60% of Asset E equally between all grandchildren and to split 40% of Asset E equally between all other added relatives.

In other embodiments, the computing system 102 modifies the beneficiaries of a client's assets based on asset distribution preferences submitted by the client (e.g., as indicated in asset distribution preferences section 908 of the profile page 900, shown in FIG. 9). In yet other embodiments, the computing system 102 modifies the beneficiaries of a client's assets based on a client's beneficiary history. In one example, the client for FIG. 8 has historically added newly born grandchildren (e.g., Grandson A, Grandson B, and Granddaughter A) to the family education fund beneficiaries portion 810 and the Asset E beneficiaries portion 812. From this, the computing system 102 determines that the client would like Grandson C to be added as a beneficiary of the family education fund and Asset E and automatically does so, as shown in portions 810 and 812. Additionally, those of skill in the art will appreciate that FIG. 8 is an example embodiment of the current beneficiaries page 800 and that in other embodiments, the computing system simply recommends to the client that the client modify his or her beneficiaries, as described above with respect to FIGS. 2 and 3.

Referring now to FIG. 9, the profile tab 902 is bolded, indicating that the client is viewing the profile page 900. As shown in FIG. 9, the profile page includes a biographical information section 904, an interests section 906, and a preferences section 908. The biographical information section 904 displays biographical information about the client, either as entered by the client (e.g., by the client pressing the “Edit” button in the biographical information section 904, which redirects the client to an information entry page) or as gathered by the financial institution computing system 102 (e.g., as discussed above with respect to the system manager circuit 250 of FIG. 2). As shown, the biographical information section 904 includes, for example, the client's name, address, and birthday. The biographical information section 904 also includes any educational institutions the client is attending or has attended (e.g., a high school, a college, a graduate school, or a professional school), any degrees the client has, and the client's current or former profession(s). The biographical information section 604 further includes the client's hometown, religious affiliation, and political affiliation. However, those of skill in the art will appreciate that the biographical information shown in the biographical information section 904 is not meant to be limiting, and, in various arrangements, the biographical information section 904 includes additional or alternative biographical information, such as information about the client's family, professional organizations the client is involved in, any boards the client sits on, and so on. In the example of FIG. 9, the biographical information section 904 shows that the financial institution computing system 102 has received biographical information on the client's name (“Client”), an educational institution the client attended (“College A”), and the client's hometown (“Hometown”).

Similarly, the interests section 906 lists the client's interests, either entered by the client (e.g., by the client pressing the “Edit” button in the biographical information section 904, which redirects the client to an information entry page) or gathered by the financial institution computing system 102 as discussed above with respect to the system manager circuit 250 of FIG. 2. The interests section 906 includes the client's hobbies, the client's professional activities, causes the client is interested in, and so on, as described above. In the example of FIG. 9, the interests section 906 shows that the computing system 102 has received three interests for the client: Interest A, Interest B, and Interest C.

As shown in FIG. 9, “College A” from the biographical information section 904 and “Interest A” and “Interest B” from the interests section 906 are each followed by an asterisk. In the example of FIG. 9, the asterisks indicate that the client has instructed the computing system 102 (e.g., by modifying the entered information through the “Edit” buttons of sections 904 and 906) that College A, Interest A, and Interest B are more important to the client for the purposes of determining potential beneficiary recommendations than the non-asterisked information. As such, the computing system 102 weighs the asterisked information more heavily when determining the potential beneficiaries for the client (as described above with respect to the beneficiary recommendation circuit 278 of FIG. 2). For example, as shown on the beneficiary recommendations page 700, the three recommended potential beneficiaries for the client (Charity C, Submitter A, and Submitter B) are presented to the client in an order based on the number of matches with the client's biographical and interests information, shown in sections 904 and 906, and based on the number of matches with the client's preferred biographical and interests information, indicated by the asterisks in sections 904 and 906. Accordingly, Submitter A, which matches with the preferred Interest B and College A associated with the client, is presented before Submitter B, which matches with the non-preferred Interest C and Hometown.

Those of skill in the art will appreciate, however, that in other embodiments the client ranks his or her biographical and/or interests information or provides to the computing system 102 other instructions on how to consider the client's information when determining potential beneficiaries for the client. Additionally, those of skill in the art will appreciate that, in various arrangements, the profile page 900 designates the client's preferred information in a different manner, such as by bolding the preferred information or including a number indicating the rank of the information, or the computing system 102 does not allow the client to designate preferred information at all.

As shown in FIG. 9, the preferences section 908 includes a subsection 910 listing the asset distribution preferences for the client and a subsection 912 listing the automatic distribution preferences for the client. In various embodiments, the client enters in his or her asset distribution preferences and automatic distribution preferences to the computing system 102 (e.g., by pressing the “Edit” button of the preferences section 908). In some embodiments, the computing system 102, additionally or alternatively, determines at least some of the asset distribution preferences for the client from the client's asset distribution history.

In the example of FIG. 9, the client's asset distribution preferences, as shown in subsection 910, include (1) increasing savings for medical expenses with declining health, (2) increasing a charity/donation percentage with declining health, (3) increasing family savings with declining health, and (4) increasing the charity/donation percentage after the client reaches the age of 65. The computing system 102 accordingly takes these asset distribution preferences into account when recommending an asset redistribution plan to the client. This is shown, for example, in the recommended asset redistribution column 512 on the asset distribution page 500. The alert section 506 indicates that the computing system 102 recommends that the client redistribute his or her assets based on the client's current health, meaning that the computing system 102 should be taking into account the client's preferences for asset distribution based on declining health (i.e., asset distribution preferences 1-3 above). In line with the client's preference to increase savings for medical expenses with declining health, the computing system 102 accordingly recommends in row 516 that the client redistribute 50% of Asset A to a medical expenses savings account and in row 518 that the client redistribute 25% of Asset B to the medical expenses savings account. In row 518, the computing system 102 also recommends that the client donate 25% of Asset B, which matches the client's preferences to increase a charity/donation percentage with declining health. Finally, in row 522, the computing system 102 recommends that the client redistribute Asset D so that 100% of Asset D is directed to the client's family education fund, which lines up with the client's preference of increasing family savings with declining health.

The client's automatic distribution preferences, as shown in subsection 912, include (1) that the computing system 102 recommend asset redistribution plans to the client but not automatically redistribute the client's assets and (2) that the computing system 102 automatically modify the client's beneficiaries in response to life events. These automatic distribution preferences are reflected on, for example, the asset distribution page 500 and the current beneficiaries page 800. As shown on the asset distribution page 500, the computing system 102 recommends a redistribution of the client's assets in column 512 but has not actually redistributed the client's assets. Instead, the asset distribution page 500 includes column 514, in which the client has the choice of whether to accept or reject the redistributions recommended by the computing system 102, which is in line with the client's automatic distribution preferences shown in subsection 912. Conversely, as shown on the current beneficiaries page 800, the alert section 804 indicates that the computing system 102 has automatically added Grandson C as a beneficiary of the family education fund and Asset E, which is also in line with the client's automatic distribution preferences shown in subsection 912.

Those of skill in the art will appreciate that the asset distribution preferences shown in subsection 910 and the automatic distribution preferences shown in subsection 912 are example preferences. In various embodiments, clients provide to the computing system 102, or the computing system 102 determines for the client based on the client's history, any number or type of preferences. Additionally, in some embodiments, the computing system 102 determines whether to modify the client's beneficiaries and/or how to redistribute the client's assets without distribution preferences provided from the client.

Those of skill in the art will further appreciate that FIGS. 5-9 illustrate example embodiments of graphical user interfaces presented to the client as part of providing estate planning prediction and recommendation services. FIGS. 5-9 are not meant to be limiting. Moreover, in certain embodiments, additional or alternative graphical user interfaces are presented to the client as part of providing estate planning predication and recommendation services.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (IC), discrete circuits, system on a chip (SOCs) circuits, etc.), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, or XNOR), resistors, multiplexers, registers, capacitors, inductors, diodes, wiring, and so on.

The “circuit” may also include one or more processors communicably coupled to one or more memories or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some embodiments, the one or more processors may be external to the apparatus, for example, the one or more processors may be a remote processor (e.g., a cloud based processor). Alternatively, or additionally, the one or more processors may be internal and/or local to the apparatus. In this regard, a given circuit or components thereof may be disposed locally (e.g., as part of a local server or a local computing system) or remotely (e.g., as part of a remote server such as a cloud based server). To that end, a “circuit” as described herein may include components that are distributed across one or more locations.

An exemplary system for implementing the overall system or portions of the embodiments might include a general purpose computing computers in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, or 3D NOR), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, or script components), in accordance with the example embodiments described herein.

It should also be noted that the term “input devices,” as described herein, may include any type of input device including, but not limited to, a keyboard, a keypad, a mouse, joystick or other input devices performing a similar function. Comparatively, the term “output device,” as described herein, may include any type of output device including, but not limited to, a computer monitor, printer, facsimile machine, or other output devices performing a similar function.

Any foregoing references to currency or funds are intended to include fiat currencies, non-fiat currencies (e.g., precious metals), and math-based currencies (often referred to as cryptocurrencies). Examples of math-based currencies include Bitcoin, Litecoin, Dogecoin, and the like.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques, with rule-based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

1. A system comprising:
a client health monitoring device associated with a health professional, the client health monitoring device configured to collect health information about a client;
a server system including a network interface configured to enable communication between the server system and the client health monitoring device via a network, the server system further including a processor and a memory having instructions stored in non-transitory machine-readable media, the instructions executable by the processor to:
receive information relating to one or more assets of a client;
receive, via the network interface, the health information from the client health monitoring device;
monitor a health status of the client using the health information;
determine that the client has poor and/or declining health based on the monitored health status; and
in response to determining that the client has poor and/or declining health:
determine a redistribution plan for the one or more assets of the client;
automatically redistribute the one or more assets of the client according to the redistribution plan; and
recommend that the client undertake one or more estate planning activities, wherein the one or more estate planning activities comprise at least one of writing or updating an estate planning legal document.

2. The system of claim 1, wherein the server system is further configured to:
receive at least one of biographical information of the client or interests information of the client; and
recommend one or more potential beneficiaries for the client based on at least one of the biographical information or the interests information.

3. The system of claim 2, wherein the server system is further configured to recommend the one or more potential beneficiaries in response to the redistribution plan comprising an increased donation percentage for at least one asset of the one or more assets of the client.

4. The system of claim 2, wherein the server system is further configured to:
receive a plurality of funding request submissions from a plurality of submitters, wherein each submitter is an individual or organization and each submission describes why the associated submitter is requesting funding; and
recommend the one or more potential beneficiaries by recommending at least one submitter as at least one of the one or more potential beneficiaries for the client, based on a commonality between at least one characteristic of each recommended submitter or the submission associated with each recommended submitter and at least one of the biographical information or the interests information of the client.

5. (canceled)

6. The system of claim 1, wherein the server system is further configured to:
determine that a life event has occurred for the client, wherein the life event comprises at least one of a birth of a relative of the client, a change in a marital status of the client, a change in a marital status of a relative of the client, or a death of a relative of the client; and
change beneficiaries of at least one asset of the client based on the determined life event.

7. The system of claim 1, wherein the server system is further configured to:
receive, from the client, distribution preferences of the client for the one or more assets of the client; and
determine the redistribution plan for the one or more assets of the client based on the health status of the client and the distribution preferences of the client.

8. (canceled)

9. A method comprising:
receiving, by a financial institution computing system, information relating to one or more assets of a client;
receiving, by the computing system, health information about the client from a client health monitoring device associated with a health professional;
monitoring, by the computing system, a health status of the client using the health information;
determining, by the computing system, that the client has poor and/or declining health based on the monitored health status; and
in response to determining that the client has poor and/or declining health:
determining, by the computing system, a redistribution plan for the one or more assets of the client;
automatically redistributing, by the computing system, the one or more assets of the client according to the redistribution plan; and
recommending, by the computing system that the client undertake one or more estate planning activities, wherein the one or more estate planning activities comprise at least one of writing or updating an estate planning legal document.

10. The method for estate planning of claim 9, the method further comprising:
receiving, by the computing system, at least one of biographical information of the client or interests information of the client; and
recommending, by the computing system, one or more potential beneficiaries for the client based on at least one of the biographical information or the interests information.

11. The method for estate planning of claim 10, wherein recommending the one or more potential beneficiaries is responsive to the redistribution plan comprising an increased donation percentage for at least one asset of the one or more assets of the client.

12. The method for estate planning of claim 10, the method further comprising:
receiving, by the computing system, a plurality of funding request submissions from a plurality of submitters, wherein each submitter is an individual or organization and each submission describes why the associated submitter is requesting funding;
wherein recommending the one or more potential beneficiaries further comprises recommending, by the computing system, at least one submitter as at least one of the one or more potential beneficiaries for the client, based on a commonality between at least one characteristic of each recommended submitter or the submission associated with each recommended submitter and at least one of the biographical information or the interests information of the client.

13. (canceled)

14. The method for estate planning of claim 9, the method further comprising:
determining, by the computing system, that a life event has occurred for the client, wherein the life event comprises at least one of a birth of a relative of the client, a change in a marital status of the client, a change in a marital status of a relative of the client, or a death of a relative of the client; and
changing, by the computing system, beneficiaries of at least one asset of the client based on the determined life event.

15. The method for estate planning of claim 9, the method further comprising:
receiving from the client, by the computing system, distribution preferences of the client for the one or more assets of the client;
wherein determining the redistribution plan further comprises determining, by the computing system, the redistribution plan for the one or more assets of the client based on the health status of the client and the distribution preferences of the client.

16. (canceled)

17. A mobile device comprising:
a network interface structured to communicate data to and from a server system associated with a financial institution;
a display device configured to present information to a client;
an input/output device structured to exchange data with the client; and
a processing circuit comprising a processor and a memory, the memory structured to store instructions that are executable by the processor and cause the processing circuit to:
receive, by the input/output device, information relating to one or more assets of the client;
transmit, by the network interface, the information relating to the one or more assets of the client to the server system;
receive, by the network interface, a redistribution plan for the one or more assets of the client from the server system, the redistribution plan based on a determination, by the server system using health information about the client received by the server system from a client health monitoring device associated with a health professional, that the client has poor and/or declining health;
capture, by the input/output device, one or more client selections relating to the redistribution plan;
transmit, by the network interface, the one or more client selections to the server system;
receive, by the network interface, a list of recommended estate planning activities from the server system, wherein the list of recommended estate planning activities comprise at least one of writing or updating an estate planning legal document and are based on the determination that the client has poor and/or declining health; and
capture, by the input/output device, one or more client selections relating to the list of recommended estate planning activities.

18. The mobile device of claim 17, wherein the processing circuit is further configured to:
receive, by the input/output device, at least one of biographical information of the client or interests information of the client;
transmit, by the network interface, the at least one of biographical information or interests information of the client to the server system; and
receive, by the network interface, an identification of one or more recommended potential beneficiaries for the client from the server system.

19. (canceled)

20. The mobile device of claim 17, wherein the processing circuit is further configured to:
receive, by the input/output device, distribution preferences of the client for the one or more assets of the client; and
transmit, by the network interface, the distribution preferences of the client to the server system.

21. The system of claim 1, wherein the client health monitoring device is a wearable device.

22. The system of claim 1, wherein the client health monitoring device is configured to be in continuous immediate proximity to the client.
